(19) United States
(12) Patent Application Publication
(10) Pub. No.: US 2007/0037820 A1
(43) Pub. Date: Feb. 15, 2007

(54) Substituted piperazines as metabotropic glutamate receptor antagonists

(75) Inventors: Louise Edwards, Toronto (CA); Abdelmalik Slassi, Mississauga (CA); Methvin Isaac, Brampton (CA); Donald McLeod, Salt Lake City, UT (US)

Correspondence Address: BIRCH, STEWART, KOLASCH & BIRCH, LLP, P.O. BOX 747, 8110 GATEHOUSE ROAD, SUITE 500 EAST, FALLS CHURCH, VA, 22040-0747, US

(73) Assignees: AstraZeneca AB, Sodertalje (SE); NPS PHARMACEUTICALS, INC., Salt Lake City, UT (US)

(21) Appl. No.: 11/498,835

(22) Filed: Aug. 4, 2006

Related U.S. Application Data
(60) Provisional application No. 60/707,946, filed on Aug. 15, 2005.

(51) Int. Cl. A61K 31/497; A61K 31/496; C07D 413/14; C07D 413/02

(52) U.S. Cl. 514/252.11; 544/364; 544/357; 544/367; 514/253.09; 514/254.04; 514/254.02

(57) ABSTRACT

The invention relates to compounds of formula I or pharmaceutically acceptable salts or solvates thereof: 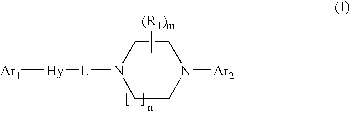
where Ar₁, Ar₂, Hy, L, R₁, m and n are as defined in the description. The invention also includes pharmaceutical compositions and uses thereof, processes for making the compounds, as well as methods for the medical treatment of mGluR5-mediated disorders.

### FIELD OF THE INVENTION

The present invention relates to a new class of compounds, to pharmaceutical formulations containing said compounds and to the use of said compounds in therapy. The present invention further relates to the process for the preparation of said compounds and to new intermediates prepa0red therein.

### BACKGROUND OF THE INVENTION

Glutamate is the major excitatory neurotransmitter in the mammalian central nervous system (CNS). Glutamate produces its effects on central neurons by binding to and thereby activating cell surface receptors. These receptors have been divided into two major classes, the ionotropic and metabotropic glutamate receptors, based on the structural features of the receptor proteins, the means by which the receptors transduce signals into the cell, and pharmacological profiles.

The metabotropic glutamate receptors (mGluRs) are G protein-coupled receptors that activate a variety of intracellular second messenger systems following the binding of glutamate. Activation of mGluRs in intact mammalian neurons elicits one or more of the following responses: activation of phospholipase C; increases in phosphoinositide (PI) hydrolysis; intracellular calcium release; activation of phospholipase D; activation or inhibition of adenyl cyclase; increases or decreases in the formation of cyclic adenosine monophosphate (cAMP); activation of guanylyl cyclase; increases in the formation of cyclic guanosine monophosphate (cGMP); activation of phospholipase A₂; increases in arachidonic acid release; and increases or decreases in the activity of voltage- and ligand-gated ion channels. Schoepp et al., Trends Pharmacol. Sci. 14:13 (1993), Schoepp, Neurochem. Int. 24:439 (1994). Pin et al., Neuropharmacology 34:1 (1995), Bordi and Ugolini, Prog. Neurobiol. 59:55 (1999).

Molecular cloning has identified eight distinct mGluR subtypes, termed mGluR1 through mGluR8. Nakanishi, Neuron 13:1031 (1994), Pin et al., Neuropharmacology 34:1 (1995), Knopfel et al., J. Med. Chem. 38:1417 (1995). Further receptor diversity occurs via expression of alternatively spliced forms of certain mGluR subtypes. Pin et al., PNAS 89:10331 (1992), Minakami et al., BBRC 199:1136 (1994), Joly et al., J. Neurosci. 15:3970 (1995).

Metabotropic glutamate receptor subtypes may be subdivided into three groups, Group I, Group II, and Group III mGluRs, based on amino acid sequence homology, the second messenger systems utilized by the receptors, and by their pharmacological characteristics. Group I mGluR comprises mGluR1, mGluR5 and their alternatively spliced variants. The binding of agonists to these receptors results in the activation of phospholipase C and the subsequent mobilization of intracellular calcium.

Neurological, Psychiatric and Pain Disorders.

Attempts at elucidating the physiological roles of Group I mGluRs suggest that activation of these receptors elicits neuronal excitation. Various studies have demonstrated that Group I mGluRs agonists can produce postsynaptic excitation upon application to neurons in the hippocampus, cerebral cortex, cerebellum, and thalamus, as well as other CNS regions. Evidence indicates that this excitation is due to direct activation of postsynaptic mGluRs, but it also has been suggested that activation of presynaptic mGluRs occurs, resulting in increased neurotransmitter release. Baskys, Trends Pharmacol. Sci. 15:92 (1992), Schoepp, Neurochem. Int. 24:439 (1994), Pin et al., Neuropharmacology 34:1(1995), Watkins et al., Trends Pharmacol. Sci. 15:33 (1994).

Metabotropic glutamate receptors have been implicated in a number of normal processes in the mammalian CNS. Activation of mGluRs has been shown to be required for induction of hippocampal long-term potentiation and cerebellar long-term depression. Bashir et al., Nature 363:347 (1993), Bortolotto et al., Nature 368:740 (1994), Aiba et al., Cell 79:365 (1994), Aiba et al., Cell 79:377 (1994). A role for mGluR activation in nociception and analgesia also has been demonstrated, Meller et al., Neuroreport 4: 879 (1993), Bordi and Ugolini, Brain Res. 871:223 (1999). In addition, mGluR activation has been suggested to play a modulatory role in a variety of other normal processes including synaptic transmission, neuronal development, apoptotic neuronal death, synaptic plasticity, spatial learning, olfactory memory, central control of cardiac activity, waking, motor control and control of the vestibulo-ocular reflex. Nakanishi, Neuron 13: 1031 (1994), Pin et al., Neuropharmacology 34:1, Knopfel et al., J. Med. Chem. 38:1417 (1995).

Further, Group I metabotropic glutamate receptors and mGluR5 in particular, have been suggested to play roles in a variety of pathophysiological processes and disorders affecting the CNS. These include stroke, head trauma, anoxic and ischemic injuries, hypoglycemia, epilepsy, neurodegenerative disorders such as Alzheimer's disease and pain. Schoepp et al., Trends Pharmacol. Sci. 14:13 (1993), Cunningham et al., Life Sci. 54:135 (1994), Hollman et al., Ann. Rev. Neurosci. 17:31 (1994), Pin et al., Neuropharmacology 34:1 (1995), Knopfel et al., J. Med. Chem. 38:1417 (1995), Spooren et al., Trends Pharmacol. Sci. 22:331 (2001), Gasparini et al. Curr. Opin. Pharmacol. 2:43 (2002), Neugebauer Pain 98:1 (2002). Much of the pathology in these conditions is thought to be due to excessive glutamate-induced excitation of CNS neurons. Because Group I mGluRs appear to increase glutamate-mediated neuronal excitation via postsynaptic mechanisms and enhanced presynaptic glutamate release, their activation probably contributes to the pathology. Accordingly, selective antagonists of Group I mGluR receptors could be therapeutically beneficial, specifically as neuroprotective agents, analgesics or anticonvulsants.

Recent advances in the elucidation of the neurophysiological roles of metabotropic glutamate receptors generally and Group I in particular, have established these receptors as promising drug targets in the therapy of acute and chronic neurological and psychiatric disorders and chronic and acute pain disorders.

Gastro Intestinal Disorders

The lower esophageal sphincter (LES) is prone to relaxing intermittently. As a consequence, fluid from the stomach can pass into the esophagus since the mechanical barrier is temporarily lost at such times, an event hereinafter referred to as “reflux”.

Gastro-esophageal reflux disease (GERD) is the most prevalent upper gastrointestinal tract disease. Current pharmacotherapy aims at reducing gastric acid secretion, or at neutralizing acid in the esophagus. The major mechanism behind reflux has been considered to depend on a hypotonic lower esophageal sphincter. However, e.g. Holloway & Dent (1990) Gastroenterol. Clin. N. Amer. 19, pp. 517-535, has shown that most reflux episodes occur during transient lower esophageal sphincter relaxations (TLESRs), i.e. relaxations not triggered by swallows. It has also been shown that gastric acid secretion usually is normal in patients with GERD.

The novel compounds according to the present invention are assumed to be useful for the inhibition of transient lower esophageal sphincter relaxations (TLESRs) and thus for treatment of gastro-esophageal reflux disorder (GERD).

The wording “TLESR”, transient lower esophageal sphincter relaxations, is herein defined in accordance with Mittal, R. K., Holloway, R. H., Penagini, R., Blackshaw, L. A., Dent, J., 1995; Transient lower esophageal sphincter relaxation. Gastroenterology 109, pp. 601-610.

The wording “reflux” is herein defined as fluid from the stomach being able to pass into the esophagus, since the mechanical barrier is temporarily lost at such times.

The wording “GERD”, gastro-esophageal reflux disease, is herein defined in accordance with van Heerwarden, M. A., Smout A. J. P. M, 2000; Diagnosis of reflux disease. Baillière's Clin. Gastroenterol. 14, pp. 759-774.

Because of their physiological and pathophysiological significance, there is a need for new potent mGluR agonists and antagonists that display a high selectivity for mGluR subtypes, particularly the Group I receptor subtype, most particularly the mGluR5

The object of the present invention is to provide compounds exhibiting an activity at metabotropic glutamate receptors (mGluRs), especially at the mGluR5 receptor.

### SUMMARY OF THE INVENTION

One embodiment of the invention relates to compounds of formula I:

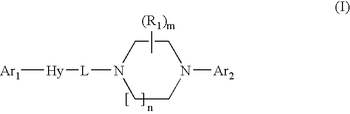

wherein:

Ar₁ and Ar₂ are independently selected, optionally substituted, aryl or heteroaryl groups, wherein the substituents are selected from the group consisting of F, Cl, Br, I, OH, nitro, C₁₋₆-alkyl, C₁₋₆-alkylhalo, OC₁₋₆-alkyl, OC₁₋₆-alkylhalo, C₂₋₆-alkenyl, C₂₋₆-alkynyl, CN, CO₂R², SR², S(O)R², SO₂R², aryl, heteroaryl, cycloalkyl and heterocycloalkyl, wherein any cyclic substituent may be further substituted with at least one substituent selected from the group consisting of F, Cl, Br, I, OH, nitro, C₁₋₆-alkyl, C₁₋₆-alkylhalo, OC₁₋₆-alkyl, OC₁₋₆-alkylhalo, C₂₋₆-alkenyl, C₂₋₆-alkynyl, CN, CO₂R², SR², S(O)R² and SO₂R²;

R₁, in each instance, is independently selected from the group consisting of F, Cl, Br, I, OH, CN, nitro, C₁₋₆-alkyl, OC₁₋₆-alkyl, C₁₋₆-alkylhalo, OC₁₋₆-alkylhalo, (CO)R², O(CO)R², O(CO)OR², CO₂R², CONR²R³, C₁₋₆-alkyleneOR², OC₂₋₆-alkyleneOR² and C₁₋₆-alkylenecyano;

R² and R³ are independently selected from the group consisting of H, C₁₋₆-alkyl, C₁₋₆-alkylhalo, C₁₋₆-alkenyl, C₂₋₆-alkynyl and cycloalkyl;

Hy is a 5-membered heterocyclic ring containing two or three heteroatoms independently selected from the group consisting of N, O and S, wherein the ring is optionally substituted with one or more substituents selected from the group consisting of F, Cl, Br, I, OH, nitro, C₁₋₆-alkyl, C₁₋₆-alkylhalo, OC₁₋₆-alkyl, OC₁₋₆-alkylhalo, CN, CO₂R², CONR²R³, SR², S(O)R² and SO₂R²;

L is selected from the group consisting of —CR⁴R⁵—, —C(O)—, —C(NR⁴)— and —C(S)—;

R⁴ and R⁵ are independently selected from the group consisting of H, C₁₋₆-alkyl, C₁₋₆-alkylhalo, C₂₋₆-alkenyl and C₂₋₆-alkynyl;

m is an integer selected from the group consisting of 0, 1, 2, 3 and 4; and

n is an integer selected from the group consisting of 1 and 2;

or a pharmaceutically-acceptable salt, hydrate, solvate, isoform, tautomer, optical isomer, or combination thereof.

Another embodiment is a pharmaceutical composition comprising as active ingredient a therapeutically effective amount of the compound according to formula I, in association with one or more pharmaceutically acceptable diluents, excipients and/or inert carriers.

Other embodiments, as described in more detail below, relate to a compound according to formula I for use in therapy, in treatment of mGluR 5 mediated disorders, in the manufacture of a medicament for the treatment of mGluR5 mediated disorders.

Still other embodiments relate to a method of treatment of mGluR5 mediated disorders, comprising administering to a mammal a therapeutically effective amount of the compound according to formula I.

In another embodiment, there is provided a method for inhibiting activation of mGlurR5 receptors, comprising treating a cell containing said receptor with an effective amount of the compound according to formula I.

### DETAILED DESCRIPTION OF THE PREFERRED EMBODIMENTS

The present invention is based upon the discovery of compounds that exhibit activity as pharmaceuticals, in particular as antagonists of metabotropic glutamate receptors. More particularly, the compounds of the present invention exhibit activity as antagonists of the mGluR5 receptor and, therefore, are useful in therapy, in particular for the treatment of neurological, psychiatric, pain, and gastrointestinal disorders associated with glutamate dysfunction.

Definitions

Unless specified otherwise within this specification, the nomenclature used in this specification generally follows the examples and rules stated in Nomenclature of Organic Chemistry, Sections A, B, C, D, E, F, and H, Pergamon Press, Oxford, 1979, which is incorporated by references herein for its exemplary chemical structure names and rules on naming chemical structures. Optionally, a name of a compound may be generated using a chemical naming program: ACD/ChemSketch, Version 5.09/September 2001, Advanced Chemistry Development, Inc., Toronto, Canada.

The term “alkyl” as used herein means a straight- or branched-chain hydrocarbon radical having from one to six carbon atoms, and includes methyl, ethyl, propyl, isopropyl, t-butyl and the like.

The term “alkenyl” as used herein means a straight- or branched-chain alkenyl radical having from two to six carbon atoms, and includes ethenyl, 1-propenyl, 1-butenyl and the like.

The term “alkynyl” as used herein means a straight- or branched-chain alkynyl radical having from two to six carbon atoms, and includes 1-propynyl (propargyl), 1-butynyl and the like.

The term “cycloalkyl” as used herein means a cyclic group (which may be unsaturated) having from three to seven carbon atoms, and includes cyclopropyl, cyclohexyl, cyclohexenyl and the like.

The term “heterocycloalkyl” as used herein means a three- to seven-membered cyclic group (which may be unsaturated) having at least one heteroatom selected from the group consisting of N, S and O, and includes piperidinyl, piperazinyl, pyrrolidinyl, tetrahydrofuranyl and the like.

The term “alkoxy” as used herein means a straight- or branched-chain alkoxy radical having from one to six carbon atoms and includes methoxy, ethoxy, propyloxy, isopropyloxy, t-butoxy and the like.

The term “halo” as used herein means halogen and includes fluoro, chloro, bromo, iodo and the like, in both radioactive and non-radioactive forms.

The term “alkylene” as used herein means a difunctional branched or unbranched saturated hydrocarbon radical having one to six carbon atoms, and includes methylene, ethylene, n-propylene, n-butylene and the like.

The term “alkenylene” as used herein means a difunctional branched or unbranched hydrocarbon radical having two to six carbon atoms and having at least one double bond, and includes ethenylene, n-propenylene, n-butenylene and the like.

The term “alkynylene” as used herein means a difunctional branched or unbranched hydrocarbon radical having two to six carbon atoms and having at least one triple bond, and includes ethynylene, n-propynylene, n-butynylene and the like.

The term “aryl” as used herein means an aromatic group having five to twelve atoms, and includes phenyl, naphthyl and the like.

The term “heteroaryl” means an aromatic group which includes at least one heteroatom selected from the group consisting of N, S and O, and includes groups and includes pyridyl, indolyl, furyl, benzofuryl, thienyl, benzothienyl, quinolyl, oxazolyl and the like.

The terms “alkylaryl”, “alkylheteroaryl” and “alkylcycloalkyl” refer to an alkyl radical substituted with an aryl, heteroaryl or cycloalkyl group, and includes 2-phenethyl, 3-cyclohexyl propyl and the like.

The term “5-membered heterocyclic ring containing two or three heteroatoms independently selected from the group consisting of N, O and S” includes aromatic and heteroaromatic rings, as well as rings which may be saturated or unsaturated, and includes isoxazolyl, oxazolyl, oxadiazolyl, pyrazolyl, thiazolyl, imidazolyl, triazolyl and the like.

The term “pharmaceutically acceptable salt” means either an acid addition salt or a basic addition salt which is compatible with the treatment of patients.

A “pharmaceutically acceptable acid addition salt” is any non-toxic organic or inorganic acid addition salt of the base compounds represented by Formula I or any of its intermediates. Illustrative inorganic acids which form suitable salts include hydrochloric, hydrobromic, sulfuric and phosphoric acid and acid metal salts such as sodium monohydrogen orthophosphate and potassium hydrogen sulfate. Illustrative organic acids which form suitable salts include the mono-, di- and tricarboxylic acids. Illustrative of such acids are, for example, acetic, glycolic, lactic, pyruvic, malonic, succinic, glutaric, fumaric, malic, tartaric, citric, ascorbic, maleic, hydroxymaleic, benzoic, hydroxybenzoic, phenylacetic, cinnamic, salicylic, 2-phenoxybenzoic, p-toluenesulfonic acid and other sulfonic acids such as methanesulfonic acid and 2-hydroxyethanesulfonic acid. Either the mono- or di-acid salts can be formed, and such salts can exist in either a hydrated, solvated or substantially anhydrous form. In general, the acid addition salts of these compounds are more soluble in water and various hydrophilic organic solvents, and generally demonstrate higher melting points in comparison to their free base forms. The selection criteria for the appropriate salt will be known to one skilled in the art Other non-pharmaceutically acceptable salts e.g. oxalates may be used for example in the isolation of compounds of Formula I for laboratory use, or for subsequent conversion to a pharmaceutically acceptable acid addition salt.

A “pharmaceutically acceptable basic addition salt” is any non-toxic organic or inorganic base addition salt of the acid compounds represented by Formula I or any of its intermediates. Illustrative inorganic bases which form suitable salts include lithium, sodium, potassium, calcium, magnesium or barium hydroxides. Illustrative organic bases which form suitable salts include aliphatic, alicyclic or aromatic organic amines such as methylamine, trimethyl amine and picoline or ammonia. The selection of the appropriate salt may be important so that an ester functionality, if any, elsewhere in the molecule is not hydrolyzed. The selection criteria for the appropriate salt will be known to one skilled in the art.

“Solvate” means a compound of Formula I or the pharmaceutically acceptable salt of a compound of Formula I wherein molecules of a suitable solvent are incorporated in a crystal lattice. A suitable solvent is physiologically tolerable at the dosage administered as the solvate. Examples of suitable solvents are ethanol, water and the like. When water is the solvent, the molecule is referred to as a hydrate.

The term “stereoisomers” is a general term for all isomers of the individual molecules that differ only in the orientation of their atoms in space. It includes mirror image isomers (enantiomers), geometric (cis/trans) isomers and isomers of compounds with more than one chiral centre that are not mirror images of one another (diastereomers).

The term “treat” or “treating” means to alleviate symptoms, eliminate the causation of the symptoms either on a temporary or permanent basis, or to prevent or slow the appearance of symptoms of the named disorder or condition.

The term “therapeutically effective amount” means an amount of the compound which is effective in treating the named disorder or condition.

The term “pharmaceutically acceptable carrier” means a non-toxic solvent, dispersant, excipient, adjuvant or other material which is mixed with the active ingredient in order to permit the formation of a pharmaceutical composition, i.e., a dosage form capable of administration to the patient. One example of such a carrier is a pharmaceutically acceptable oil typically used for parenteral administration.

Compounds

Compounds of the invention conform generally to formula I:

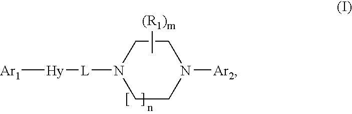

wherein Ar, Hy, L, R₁, m and n are defined hereinabove.

In one embodiment, Ar₁ is an optionally-substituted phenyl group; illustrative substituents may be selected from the group consisting of F, Cl, Br, nitro, C₁₋₆-alkyl, C₁₋₆-alkylhalo, OC₁₋₆-alkyl, OC₁₋₆-alkylhalo, and CN.

In another embodiment, Ar₂ is an optionally-substituted pyridyl group, for example a 2-pyridyl group; illustrative substituents may be selected from the group consisting of F, Cl, Br, nitro, C₁₋₆-alkyl, C₁₋₆-allylhalo, OC₁₋₆-alkyl, OC₁₋₆-alkylhalo, and CN.

In one embodiment Hy is an oxazole group; in another it is an isoxazole group; in yet others it is an oxadiazole group or a triazole group.

In one embodiment L is a —CH₂— group; in another it is a —CH(Me)— group; in yet another it is a C(O) group.

In still another embodiment, R₁ is H or C₁₋₆-alkyl.

In one embodiment, n is 1; in another n is 2.

In yet another embodiment, m is 0; in others m is 1 or 2.

It will be understood by those of skill in the art that when compounds of the present invention contain one or more chiral centers, the compounds of the invention may exist in, and be isolated as, enantiomeric or diastereomeric forms, or as a racemic mixture. The present invention includes any possible enantiomers, diastereomers, racemates or mixtures thereof, of a compound of formula I. The optically active forms of the compound of the invention may be prepared, for example, by chiral chromatographic separation of a racemate or chemical or enzymatic resolution methodology, by synthesis from optically active starting materials or by asymmetric synthesis based on the procedures described thereafter.

It will also be appreciated by those of skill in the art that certain compounds of the present invention may exist as geometrical isomers, for example E and Z isomers of alkenes. The present invention includes any geometrical isomer of a compound of formula I. It will further be understood that the present invention encompasses tautomers of the compounds of formula I.

It will also be understood by those of skill in the art that certain compounds of the present invention may exist in solvated, for example hydrated, as well as unsolvated forms. It will further be understood that the present invention encompasses all such solvated forms of the compounds of formula I.

Within the scope of the invention are also salts of the compounds of formula I. Generally, pharmaceutically acceptable salts of compounds of the present invention are obtained using standard procedures well known in the art, for example, by reacting a sufficiently basic compound, for example an alkyl amine with a suitable acid, for example, HCl or acetic acid, to afford a salt with a physiologically acceptable anion. It is also possible to make a corresponding alkali metal (such as sodium, potassium, or lithium) or an alkaline earth metal (such as a calcium) salt by treating a compound of the present invention having a suitably acidic proton, such as a carboxylic acid or a phenol, with one equivalent of an alkali metal or alkaline earth metal hydroxide or alkoxide (such as the ethoxide or methoxide), or a suitably basic organic amine (such as choline or meglumine) in an aqueous medium, followed by conventional purification techniques. Additionally, quaternary ammonium salts can be prepared by the addition of alkylating agents, for example, to neutral amines.

In one embodiment of the present invention, the compound of formula I may be converted to a pharmaceutically acceptable salt or solvate thereof, particularly, an acid addition salt such as a hydrochloride, hydrobromide, phosphate, acetate, fumarate, maleate, tartrate, citrate, methanesulphonate or p-toluenesulphonate.

Specific examples of the present invention include the following compounds, their pharmaceutically acceptable salts, hydrates, solvates, optical isomers, and combinations thereof:

| Example | Compound | Name |
|---|---|---|
| 15.1 | 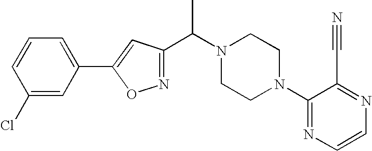 | 3-(4-{1-[5-(3- chlorophenyl)isoxazol-3- yl]ethyl}piperazin-1- yl)pyrazine-2-carbonitrile |
| 15.2 | 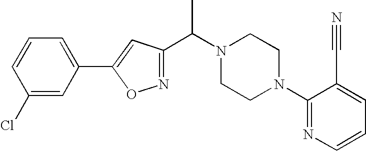 | 2-(4-{1-[5-(3- chlorophenyl)isoxazol-3- yl]ethyl}piperazin-1-yl) nicotinonitrile |
| 15.3 | 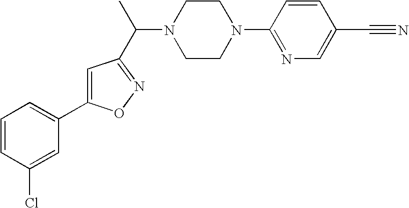 | 6-(4-{1-[5-(3- chlorophenyl)isoxazol-3- yl]ethyl}piperazin-1-yl) nicotinonitrile |
| 15.4 | 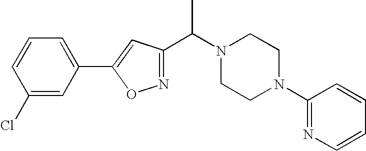 | 1-{1-[5-(3- chlorophenyl)isoxazol-3- yl]ethyl}-4-pyridin-2- ylpiperazine |
| 15.5 | 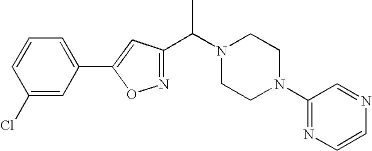 | 2-(4-{1-[5-(3- chlorophenyl)isoxazol-3- yl]ethyl}piperazin-1- yl)pyrazine |
| 15.6 | 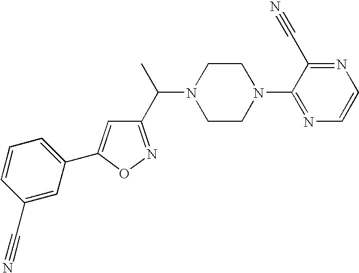 | 3-(4-{1-[5-(3- cyanophenyl)isoxazol-3- yl]ethyl}piperazin-1- yl)pyrazine-2-carbonitrile |
| 15.7 | 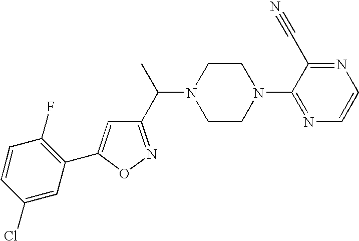 | 3-(4-{1-[5-(5-chloro-2- fluorophenyl)isoxazol-3- yl]ethyl}piperazin-1-yl) pyrazine-2-carbonitrile |
| 15.8 | 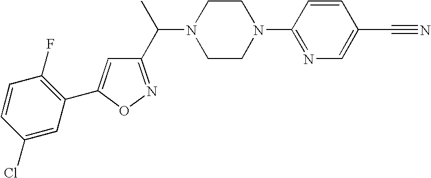 | 6-(4-{1-[5-(5-chloro-2- fluorophenyl)isoxazol-3- yl]ethyl}piperazin-1-yl) nicotinonitrile |
| 15.9 | 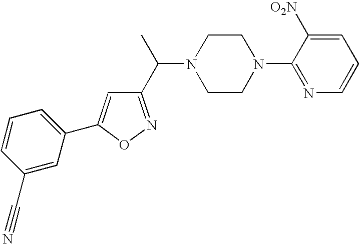 | 3-(3-{1-[4-(3-nitropyridin- 2-yl)piperazin-1- yl]ethyl}isoxazol-5-yl) benzonitrile |
| 15.10 | 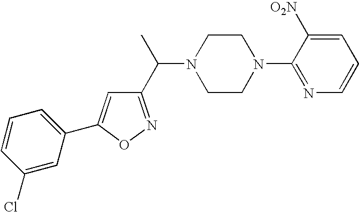 | 1-{1-[5-(3- chlorophenyl)isoxazol-3- yl]ethyl}-4-(3- nitropyridin-2-yl) piperazine |
| 15.11 | 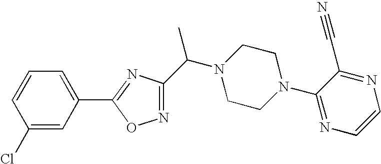 | 3-(4-{1-[5-(3- chlorophenyl)-1,2,4- oxadiazol-3- yl]ethyl}piperazin-1-yl) pyrazine-2-carbonitrile |
| 15.12 | 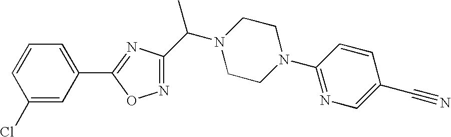 | 6-(4-{1-[5-(3- chlorophenyl)-1,2,4- oxadiazol-3- yl]ethyl}piperazin-1-yl) nicotinonitrile |
| 15.13 | 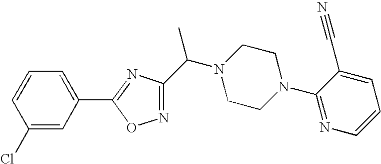 | 2-(4-{1-[5-(3- chlorophenyl)-1,2,4- oxadiazol-3- yl]ethyl}piperazin-1-yl) nicotinonitrile |
| 15.14 | 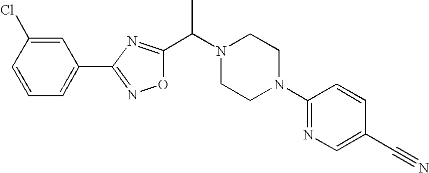 | 6-(4-{1-[3-(3- chlorophenyl)-1,2,4- oxadiazol-5- yl]ethyl}piperazin-1-yl) nicotinonitrile |
| 15.15 | 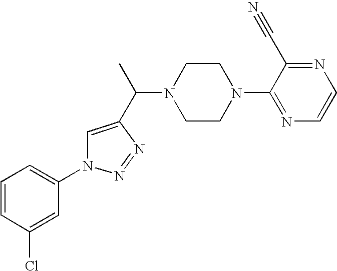 | 3-(4-{1-[1-(3- chlorophenyl)-1H-1,2,3- triazol-4- yl]ethyl}piperazin-1-yl) pyrazine-2-carbonitrile |
| 15.16 | 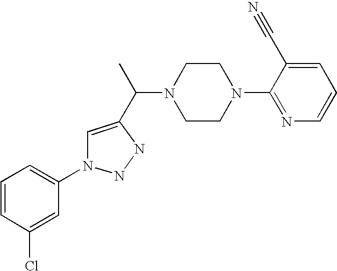 | 2-(4-{1-[1-(3- chlorophenyl)-1H-1,2,3- triazol-4- yl]ethyl}piperazin-1-yl) nicotinonitrile |
| 15.17 | 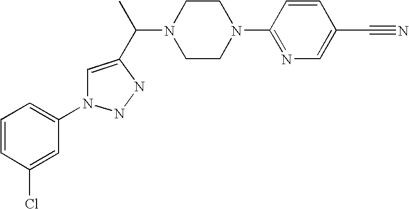 | 6-(4-{1-[1-(3- chlorophenyl)-1H-1,2,3- triazol-4- yl]ethyl}piperazin-1-yl) nicotinonitrile |
| 15.18 | 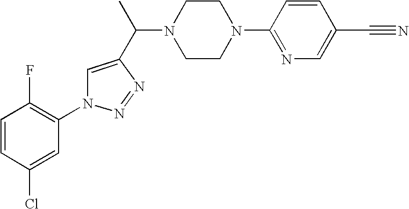 | 6-(4-{1-[1-(5-chloro-2- fluoro phenyl)-1H-1,2,3- triazol-4-yl]ethyl}piperazine-1-yl) nicotinonitrile |
| 15.19 | 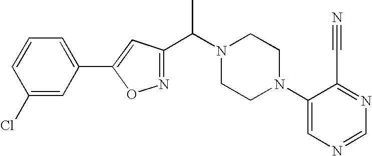 | 5-(4-{1-[5-(3- chlorophenyl)isoxazol-3- yl]ethyl}piperazin-1- yl)pyrimidine-4- carbonitrile |
| 15.20 | 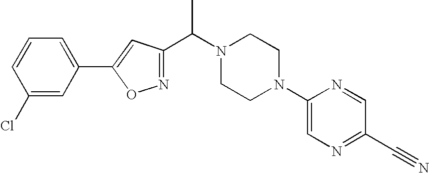 | 5-(4-{1-[5-(3- chlorophenyl)isoxazol-3- yl]ethyl}piperazin-1- yl)pyrazine-2-carbonitrile |
| 15.21 | 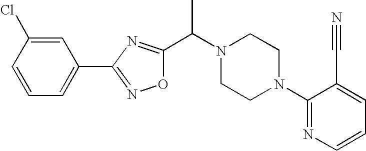 | 2-(4-{1-[3-(3- chlorophenyl)-1,2,4- oxadiazol-5- yl]ethyl}piperazin-1-yl) nicotinonitrile |
| 15.22 | 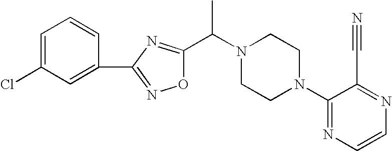 | 3-(4-{1-[3-(3- chlorophenyl)-1,2,4- oxadiazol-5- yl]ethyl}piperazin-1-yl) pyrazine-2-carbonitrile |
| 16.1 | 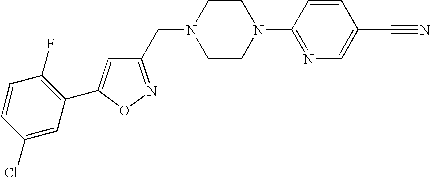 | 6-(4-{[5-(5-chloro-2- fluorophenyl) isoxazol-3- yl]methyl}piperazin-1-yl) nicotinonitrile |
| 16.2 | 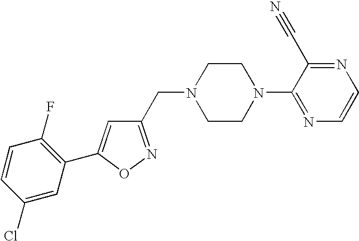 | 3-(4-{[5-(5-chloro-2- fluorophenyl) isoxazol-3- yl]methyl}piperazin-1-yl) pyrazine-2-carbonitrile |
| 17.1 | 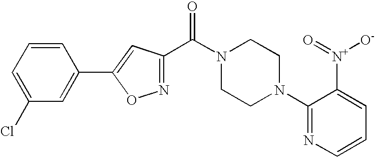 | 1-{[5-(3- chlorophenyl)isoxazol-3- yl]carbonyl}-4-(3- nitropyridin-2- yl)piperazine |
| 17.2 | 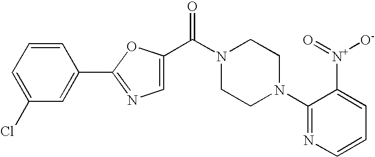 | 1-{[2-(3-chlorophenyl)- 1,3-oxazol-5-yl]carbonyl}- 4-(3-nitropyridin-2- yl)piperazine |

Pharmaceutical Composition

The compounds of the present invention may be formulated into conventional pharmaceutical composition comprising a compound of formula I, or a pharmaceutically acceptable salt or solvate thereof, in association with a pharmaceutically acceptable carrier or excipient. The pharmaceutically acceptable carriers can be either solid or liquid. Solid form preparations include, but are not limited to, powders, tablets, dispersible granules, capsules, cachets, and suppositories.

A solid carrier can be one or more substances, which may also act as diluents, flavoring agents, solubilizers, lubricants, suspending agents, binders, or tablet disintegrating agents. A solid carrier can also be an encapsulating material.

In powders, the carrier is a finely divided solid, which is in a mixture with the finely divided compound of the invention, or the active component In tablets, the active component is mixed with the carrier having the necessary binding properties in suitable proportions and compacted in the shape and size desired.

For preparing suppository compositions, a low-melting wax such as a mixture of fatty acid glycerides and cocoa butter is first melted and the active ingredient is dispersed therein by, for example, stirring. The molten homogeneous mixture is then poured into convenient sized moulds and allowed to cool and solidify.

Suitable carriers include, but are not limited to, magnesium carbonate, magnesium stearate, talc, lactose, sugar, pectin, dextrin, starch, tragacanth, methyl cellulose, sodium carboxymethyl cellulose, low-melting wax, cocoa butter, and the like.

The term composition is also intended to include the formulation of the active component with encapsulating material as a carrier providing a capsule in which the active component (with or without other carriers) is surrounded by a carrier which is thus in association with it. Similarly, cachets are included.

Tablets, powders, cachets, and capsules can be used as solid dosage forms suitable for oral administration.

Liquid form compositions include solutions, suspensions, and emulsions. For example, sterile water or water propylene glycol solutions of the active compounds may be liquid preparations suitable for parenteral administration. Liquid compositions can also be formulated in solution in aqueous polyethylene glycol solution.

Aqueous solutions for oral administration can be prepared by dissolving the active component in water and adding suitable colorants, flavoring agents, stabilizers, and thickening agents as desired. Aqueous suspensions for oral use can be made by dispersing the finely divided active component in water together with a viscous material such as natural synthetic gums, resins, methyl cellulose, sodium carboxymethyl cellulose, and other suspending agents known to the pharmaceutical formulation art. Exemplary compositions intended for oral use may contain one or more coloring, sweetening, flavoring and/or preservative agents.

Depending on the mode of administration, the pharmaceutical composition will include from about 0.05% w (percent by weight) to about 99% w, more particularly, from about 0.10% w to 50% w, of the compound of the invention, all percentages by weight being based on the total weight of the composition.

A therapeutically effective amount for the practice of the present invention can be determined by one of ordinary skill in the art using known criteria including the age, weight and response of the individual patient, and interpreted within the context of the disease which is being treated or which is being prevented.

Medical Use

It has been found that the compounds according to the present invention, exhibit a high degree of potency and selectivity for individual metabotropic glutamate receptor (mGluR) subtypes. Accordingly, the compounds of the present invention are expected to be useful in the treatment of conditions associated with excitatory activation of mGluR5 and for inhibiting neuronal damage caused by excitatory activation of mGluR5. The compounds may be used to produce an inhibitory effect of mGluR5 in mammals, including man.

The Group I mGluR receptors including mGluR5 are highly expressed in the central and peripheral nervous system and in other tissues. Thus, it is expected that the compounds of the invention are well suited for the treatment of mGluR5-mediated disorders such as acute and chronic neurological and psychiatric disorders, gastrointestinal disorders, and chronic and acute pain disorders.

The invention relates to compounds of Formula I, as defined hereinbefore, for use in therapy.

The invention relates to compounds of Formula I, as defined hereinbefore, for use in treatment of mGluR5-mediated disorders.

The invention relates to compounds of Formula I, as defined hereinbefore, for use in treatment of Alzheimer's disease senile dementia, AIDS-induced dementia, Parkinson's disease, amylotropic lateral sclerosis, Huntington's Chorea, migraine, epilepsy, schizophrenia, depression, anxiety, acute anxiety, ophthalmological disorders such as retinopathies, diabetic retinopathies, glaucoma, auditory neuropathic disorders such as tinnitus, chemotherapy induced neuropathies, post-herpetic neuralgia and trigeminal neuralgia, tolerance, dependency, Fragile X, autism, mental retardation, schizophrenia and Down's Syndrome.

The invention relates to compounds of Formula I, as defined above, for use in treatment of pain related to migraine, inflammatory pain, neuropathic pain disorders such as diabetic neuropathies, arthritis and rheumatoid diseases, low back pain, post-operative pain and pain associated with various conditions including cancer, angina, renal or billiary colic, menstruation, migraine and gout

The invention relates to compounds of Formula I as defined hereinbefore, for use in treatment of stroke, head trauma, anoxic and ischemic injuries, hypoglycemia, cardiovascular diseases and epilepsy.

The present invention relates also to the use of a compound of Formula I as defined hereinbefore, in the manufacture of a medicament for the treatment of mGluR Group I receptor-mediated disorders and any disorder listed above.

One embodiment of the invention relates to the use of a compound according to Formula I in the treatment of gastrointestinal disorders.

Another embodiment of the invention relates to the use of a Formula I compound for the manufacture of a medicament for inhibition of transient lower esophageal sphincter relaxations, for the treatment of GERD, for the prevention of G.I. reflux, for the treatment regurgitation, for treatment of asthma, for treatment of laryngitis, for treatment of lung disease, for the management of failure to thrive, for the treatment of irritable bowel disease (IBS) and for the treatment of functional dyspepsia (FD).

The invention also provides a method of treatment of mGluR5-mediated disorders and any disorder listed above, in a patient suffering from, or at risk of, said condition, which comprises administering to the patient an effective amount of a compound of Formula I, as hereinbefore defined.

The dose required for the therapeutic or preventive treatment of a particular disorder will necessarily be varied depending on the host treated, the route of administration and the severity of the illness being treated.

In the context of the present specification, the term “therapy” and “treatment” includes prevention or prophylaxis, unless there are specific indications to the contrary. The terms “therapeutic” and “therapeutically” should be construed accordingly.

In this specification, unless stated otherwise, the term “antagonist” and “inhibitor” shall mean a compound that by any means, partly or completely, blocks the transduction pathway leading to the production of a response by the ligand.

The term “disorder”, unless stated otherwise, means any condition and disease associated with metabotropic glutamate receptor activity.

Non-Medical Use

In addition to their use in therapeutic medicine, the compounds of Formula I, as well as salts and hydrates of such compounds, are useful as pharmacological tools in the development and standardization of in vitro and in vivo test systems for the evaluation of the effects of inhibitors of mGluR related activity in laboratory animals such as cats, dogs, rabbits, monkeys, rats and mice, as part of the search for new therapeutics agents.

Process of Preparation

Another aspect of the present invention provides processes for preparing compounds of Formula I, or salts or hydrates thereof. Processes for the preparation of the compounds in the present invention are described herein. The synthesis of certain heterocycles Hy (for example oxazoles, isoxazoles and 1,2,4-oxadiazoles) is described in published PCT applications WO04014881, WO04014370 and WO05080379, the salient details of which are shown below.

Throughout the following description of such processes it is to be understood that, where appropriate, suitable protecting groups will be added to, and subsequently removed from, the various reactants and intermediates in a manner that will be readily understood by one skilled in the art of organic synthesis. Conventional procedures for using such protecting groups as well as examples of suitable protecting groups are described, for example, in “Protective Groups in Organic Synthesis”, T. W. Green, P. G. M. Wuts, Wiley-Interscience, New York, (1999). It also is to be understood that a transformation of a group or substituent into another group or substituent by chemical manipulation can be conducted on any intermediate or final product on the synthetic path toward the final product, in which the possible type of transformation is limited only by inherent incompatibility of other functionalities carried by the molecule at that stage to the conditions or reagents employed in the transformation. Such inherent incompatibilities, and ways to circumvent them by carrying out appropriate transformations and synthetic steps in a suitable order, will be readily understood to the one skilled in the art of organic synthesis. Examples of transformations are given below, and it is to be understood that the described transformations are not limited only to the generic groups or substituents for which the transformations are exemplified. References and descriptions on other suitable transformations are given in “Comprehensive Organic Transformations—A Guide to Functional Group Preparations” R. C. Larock, VHC Publishers, Inc. (1989). References and descriptions of other suitable reactions are described in textbooks of organic chemistry, for example, “Advanced Organic Chemistry”, March, 4th ed. McGraw Hill (1992) or, “Organic Synthesis”, Smith, McGraw Hill, (1994). Techniques for purification of intermediates and final products include for example, normal and reversed phase chromatography on column or rotating plate, recrystallisation, distillation and liquid-liquid or solid-liquid extraction, which will be readily understood by the one skilled in the art. The definitions of substituents and groups are as in formula I except where defined differently. The term “room temperature” and “ambient temperature” shall mean, unless otherwise specified, a temperature between 16 and 25° C.

Preparation of Intermediates

a) Formation of Oxadiazoles of Formula (iv)

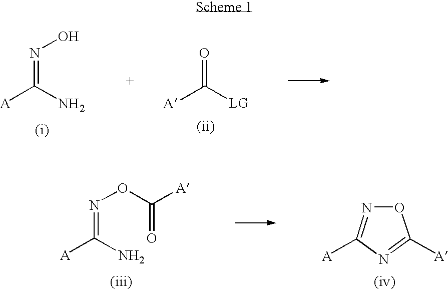

As shown in Scheme 1, a compound of formula (iv), wherein A and A′ are independently selected from the group consisting of Ar₁ and L-LG² (wherein LG² is a leaving group such as chloro or mesylate) may be prepared through cyclization of a compound of formula (iii), which in turn may be formed from a suitably activated compound of formula (ii) with a compound of formula (i).

Compounds of formula (i) may be prepared from a suitable nitrile, or from a suitably substituted cyanamide by addition of hydroxylamine, for example as the hydrochloride salt, in a suitable solvent such as, methanol, ethanol, water, dioxane or mixture thereof, using an appropriate base such as hydroxide, carbonate, acetate, or pyridine.

The compound of formula (ii) may be activated in the following non-limiting ways: i) as the acid chloride formed from the acid using a suitable reagent such as oxalyl chloride or thionyl chloride; ii) as an anhydride or mixed anhydride formed from treatment with a reagent such as alkyl chloroformate; iii) using traditional methods to activate acids in amide coupling reactions such as EDCI with HOBt or uronium salts like HBTU; iv) as an alkyl ester when the hydroxyamidine is deprotonated using a strong base like sodium tert-butoxide or sodium hydride in a solvent such as ethanol or toluene at elevated temperatures (80-110° C.).

This transformation of compounds (i) and (ii) into compounds of type (iv) may be performed as two consecutive steps via an isolated intermediate of type (iii), as described above, or the cyclization of the intermediate formed in situ may occur spontaneously during the ester formation. The formation of ester (iii) may be accomplished using an appropriate aprotic solvent such as DCM, tetrahydrofuran, N,N-dimethylformamide or toluene, with optionally an appropriate organic base such as triethylamine, diisopropylethylamine and the like or an inorganic base such sodium bicarbonate or potassium carbonate. The cyclization of compounds of formula (iii) to form an oxadiazole may be carried out on the crude ester with evaporation and replacement of the solvent with a higher boiling solvent such as DMF or with aqueous extraction to provide a semi-purified material or with material purified by standard chromatographic methods. The cyclization may be accomplished by heating conventionally or by microwave irradiation (100-180° C.), in a suitable solvent such as pyridine or N,N-dimethylformamide or using a lower temperature method employing reagents like tetrabutylammonium fluoride in tetrahydrofuran or by any other suitable known literature method.

Further examples of the above described reactions can be found in Poulain et al., Tetrahedron Lett., (2001), 42, 1495-98, Ganglott et al., Tetrahedron Lett., (2001), 42, 1441-43, and Mathvink et al, Bioorg. Med. Chem. Lett. (1999), 9, 1869-74, which are hereby included as references

b) Formation of Isoxazoles of Formula (ix)

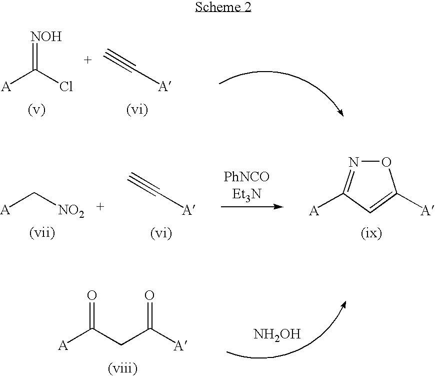

As shown in Scheme 2, a compound of formula (ix), wherein A and A are independently selected from the group consisting of Ar₁ and L-LG² (wherein LG² is a leaving group such as chloro or mesylate) may be prepared by a 1,3-dipolar cycloaddition between compounds of formula (v) and (vi) under basic conditions using a suitable base such as sodium bicarbonate or triethylamine at suitable temperatures (0° C.-100° C.) in solvents such as toluene. Synthesis of compounds of type (v) has previously been described in the literature, e.g. Kim, Jae Nyoung; Ryu, Eung K; J. Org. Chem. (1992), 57, 6649-50. 1,3-Dipolar cycloaddition with acetylenes of type (vi) can also be effected using substituted nitromethanes of type (vii) via activation with an electrophilic reagent such as PhNCO in the presence of a base such as triethylamine at elevated temperatures (50-100° C.). Li, C—S.; Lacasse, E.; Tetrahedron Lett. (2002) 43; 3565-3568. Several compounds of type (vi) are commercially available, or may be synthesized by standard methods as known by one skilled in the art.

Alternatively, compounds of formula (viii), which are available from a Claisen condensation of a methyl ketone and an ester using basic conditions using such bases as sodium hydride or potassium tert-butoxide, may yield compounds of formula (ix) via condensation and subsequent cyclization using hydroxylamine, for example in the form of the hydrochloric acid salt, at elevated temperatures (60-120° C.).

It is understood that for both methods subsequent functional group transformations may be necessary. In the case of an ester group, these transformations may include, but is not limited to either of following three procedures: a) Complete reduction using a suitable reducing agent such as LAH in solvents such as THF. b) Partial reduction using a suitable selective reducing agent such as DIBAL followed by alkylation with an alkylhalide. c) Alkylation using an alkylmetal reagent such as an alkyl magnesium halide in solvents such as toluene or THF, followed by reduction with for example sodium borohydride in methanol.

c) Formation of 1,3-Oxazoles of Formula (xii) and (xv)

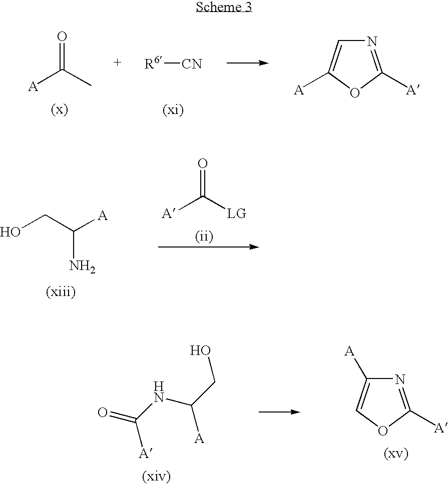

As shown in Scheme 3, a compound of formula (XII), wherein A and A are independently selected from the group consisting of Ar₁ and L-LG² (wherein LG² is a leaving group such as chloro or mesylate) may be prepared by the reaction of compounds of formula (x) and (xi) in the presence of in situ generated Tl(OTf)3 under acidic conditions according to the procedure of Lee and Hong; Tetrahedron Lett., (1997), 38, 8959-60.

Alternatively isomer (xv) is available from reaction of compounds of formula (ii) and (xiii) are reacted as described above for formula (iv) to give an intermediate of formula (xiv). Such an intermediate may give the required oxazole by cyclodehydration with Deoxo-Fluor® to generate the oxazoline followed by dehydrogenation using BrCCl₃ in the same reaction pot. Phillips, A. J.; Uto, Y.; Wipf, P.; Reno, M. J. and Williams, D. R., Organic Letters, (2000) 2, 1165-8.

d) Formation of 1,2,3-Triazoles

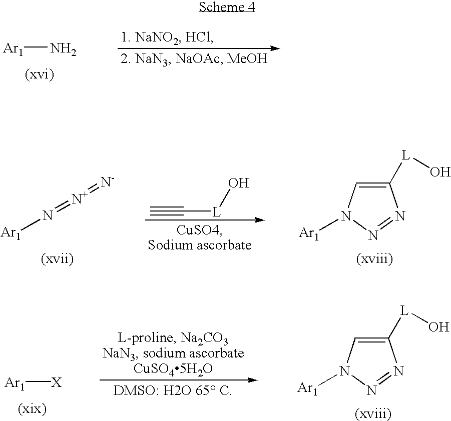

With reference to Scheme 4,1-aryl-1H-1,2,3-triazole-derivatives (xviii) may be prepared from commercially available anilines (xvi) by initial diazotization followed by conversion of the diazonium salt to the corresponding azide (xvii) using NaN₃. The aryl azide may then be cyclized with propargyl alcohol in a regiospecific manner using catalytic CuSO₄ to afford the [1,2,3]triazole alcohol intermediate (xviii) (See Rostovtsev, V. V., Green, L. G., Fokin, V. V., Sharpless, K. B.: Angew., Chem. Intl. Ed. 2002, 41, 14, 2596-2599.) The azide may also be formed in situ from the aryl iodide or bromide (xix) according to the procedure of Organic Letters 2004, Vol. 6, No. 22, 3897-3899 by heating a mixture of aryl iodide or bromide (xix), propargyl alcohol, L-proline, sodium carbonate, sodium azide, sodium ascorbate and copper sulfate pentahydrate in 9:1 DMSO:H₂O at 65° C.

Preparation of Final Compounds

Compounds of Formula I may be prepared by treatment of the above intermediates with a nucleophile under Sn2 conditions. Typically, an intermediate in which leaving group LG is a mesylate or chloride is treated with, for example, an appropriately-substituted aryl piperazine under mildly basic conditions.

Alternatively, compounds of Formula I may be prepared by reductive amination of, for example, an appropriately-substituted aryl piperazine with an intermediate in which L-LG² represents an aldehyde group.

Compounds of Formula I may also be prepared by EDCI coupling of, for example, an appropriately-substituted aryl piperazine with an intermediate in which L-LG² represents a CO₂H group.

The invention is further illustrated by way of the following examples, which are intended to elaborate several embodiments of the invention. These examples are not intended to, nor are they to be construed to, limit the scope of the invention. It will be clear that the invention may be practiced otherwise than as particularly described herein. Numerous modifications and variations of the present invention are possible in view of the teachings herein and, therefore, are within the scope of the invention.

General Methods

Abbreviations

BOC tert-butoxycarbonyl

BSA Bovine Serum Albumin

CCD Charge Coupled Device

DBU 1,8-diazabicyclo[5.4.0]undec-7-ene

DCM dichloromethane

DHPG 3,5-dihydroxyphenylglycine;

DIBAL diisobutylaluminum hydride

DMF N,N-dimethylformamide

DMSO dimethyl sulfoxide

FLIPR Fluorometric Imaging Plate reader

GC/MS gas chromatograph coupled mass spectroscopy

GHEK Human Embryonic Kidney expressing Glutamate Transporter

HEPES 4-2-hydroxyethyl)-1-piperazineethanesulfonic acid (buffer)

IP₃ inositol triphosphate

MCPBA 3-chloroperbenzoic acid

MeOH methanol

NMP N-Methylpyrrolidinone

NMR nuclear magnetic resonance

PCC pyridinium chlorochromate

ppm parts per million

RT room temperature

SPE solid phase extraction

TFA trifluoroacetic acid

THF tetrahydrofuran

TLC thin layer chromatography

All starting materials are commercially available or earlier described in the literature. Synthesis of certain heterocycles Hy are described in published PCT applications WO04014881, WO04014370 and WO05080379.

The ¹H and ¹³C NMR spectra were recorded either on Bruker 300, Bruker DPX400 or Varian +400 spectrometers operating at 300, 400 and 400 MHz for ¹H NMR respectively, using TMS or the residual solvent signal as reference, in deuterated chloroform as solvent unless otherwise indicated. All reported chemical shifts are in ppm on the delta-scale, and the fine splitting of the signals as appearing in the recordings (s: singlet, br s: broad singlet, d: doublet, t: triplet, q: quartet, m: multiplet). Unless otherwise indicated, in the tables below ¹H NMR data was obtained at 300 MHz, using CDCl₃ as the solvent.

Analytical in line liquid chromatography separations followed by mass spectra detections, were recorded on a Waters LCMS consisting of an Alliance 2795 (LC) and a ZQ single quadropole mass spectrometer. The mass spectrometer was equipped with an electrospray ion source operated in a positive and/or negative ion mode. The ion spray voltage was ±3 kV and the mass spectrometer was scanned from m/z 100-700 at a scan time of 0.8 s. To the column, X-Terra MS, Waters, C8, 2.1×50 mm, 3.5 mm, was applied a linear gradient from 5% to 100% acetonitrile in10 mM ammonium acetate (aq.), or in 0.1% TFA (aq.).

Purification of products were also done using Chem Elut Extraction Columns (Varian, cat #1219-8002), Mega BE-SI (Bond Elut Silica) SPE Columns (Varian, cat #12256018; 12256026; 12256034), or by flash chromatography in silica-filled glass columns.

Microwave heating was performed in an Emrys Optimizer from Biotage/Personal Chemistry or a Smith Synthesizer Single-mode microwave cavity producing continuous irradiation at 2450 MHz (Personal Chemistry AB, Uppsala, Sweden).

The pharmacological properties of the compounds of the invention can be analyzed using standard assays for functional activity. Examples of glutamate receptor assays are well known in the art as described in for example Aramori et al., Neuron 8:757 (1992), Tanabe et al., Neuron 8:169 (1992), Miller et al., J. Neuroscience 15: 6103 (1995), Balazs, et al., J. Neurochemistry 69:151 (1997). The methodology described in these publications is incorporated herein by reference. Conveniently, the compounds of the invention can be studied by means of an assay that measures the mobilization of intracellular calcium, [Ca²⁺]ᵢ in cells expressing mGluR5.

Intracellular calcium mobilization was measured by detecting changes in fluorescence of cells loaded with the fluorescent indicator fluo-3. Fluorescent signals were measured using the FLIPR system (Molecular Devices). A two addition experiment was used that could detect compounds that either activate or antagonize the receptor.

For FLIPR analysis, cells expressing human mGluR5d were seeded on collagen coated clear bottom 96-well plates with black sides and analysis of [Ca²⁺]ᵢ mobilization was done 24 h. after seeding.

FLIPR experiments were done using a laser setting of 0.800 W and a 0.4 second CCD camera shutter speed. Each FLIPR experiment was initiated with 160 μL of buffer present in each well of the cell plate. After each addition of the compound, the fluorescence signal was sampled 50 times at 1 second intervals followed by 3 samples at 5 second intervals. Responses were measured as the peak height of the response within the sample period.

EC₅₀ and IC₅₀ determinations were made from data obtained from 8-point concentration response curves (CRC) performed in duplicate. Agonist CRC were generated by scaling all responses to the maximal response observed for the plate. Antagonist block of the agonist challenge was normalized to the average response of the agonist challenge in 14 control wells on the same plate.

We have validated a secondary functional assay for mGluR5d based on Inositol Phosphate (IP₃) turnover. IP₃ accumulation is measured as an index of receptor mediated phospholipase C turnover. GHEK cells stably expressing the human mGluR5d receptors were incubated with [3H] myo-inositol overnight, washed three times in HEPES buffered saline and pre-incubated for 10 min. with 10 mM LiCl. Compounds (agonists) were added and incubated for 30 min. at 37° C. Antagonist activity was determined by pre-incubating test compounds for 15 min., then incubating in the presence of glutamate (80 μM) or DHPG (30 μM) for 30 min. Reactions were terminated by the addition of perchloric acid (5%). Samples were collected and neutralized, and inositol phosphates were separated using Gravity-Fed Ion-Exchange Columns.

A detailed protocol for testing the compounds of the invention is provided below in Pharmaceutical Examples.

### EXAMPLE 1

### 5-Bromopyrimidine-4-carbonitrile

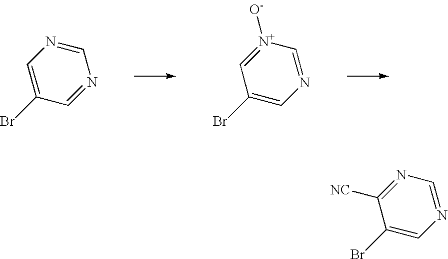

i) 5-Bromopyrimidine (50 mmol) and MCPBA (57.5 mmol) were heated under reflux in chloroform (100 mL) for 8 h. The reaction mixture was concentrated to dryness under reduced vacuum. The solid was taken up in saturated bicarbonate (100 mL) and extracted with DCM (3×100 mL). The organic layer was dried with magnesium sulfate, filtered, and evaporated to dryness under reduced vacuum. The solid was triturated with diethyl ether (30 mL plus 10 mL rinse) to give 5-bromopyrimidine-1-oxide (23%).

ii) 5-Bromopyrimidine-1-oxide (0.46 mmol) was treated with trimethylsilylcyanide (0.92 mmol) and triethylamine (1.84 mmol) in acetonitrile (50 mL) at ambient temperature for 2 h. The crude product was concentrated and purified by chromatography (silica gel, hexanes/ethyl acetate) to yield the title compound (0.46 g, 20%). ¹H NMR (CDCl₃) δ (ppm): 9.27 (s, 1H); 9.09 (s, 1H).

### EXAMPLE 2

### 5-bromopyrazine-2-carbonitrile

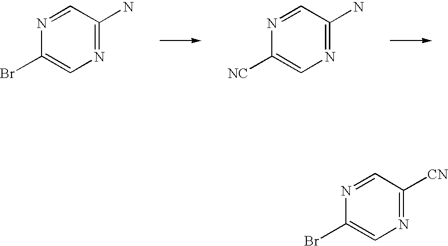

i) Tetrakis (triphenyl phosphine) palladium(0) (10 mg) was added to a mixture of 5-bromopyrazin-2-amine (0.575 mmol), potassium cyanide (1.15 mmol), copper (I) iodide (0.575 mmol) and 18-crown-6 (20 mg) in dry DMF (1 mL) in a screw top reaction vessel under nitrogen atmosphere, and the mixture was heated with stirring at 200° C. for 1 h. After cooling, water (5 mL) was added and the product was extracted with chloroform (2×) to give 5-aminopyrazine-2-carbonitrile after purification by chromatography (silica, hexane:ethyl acetate) (0.208 mmol, 36%).

ii) 5-Aminopyrazine-2-carbonitrile (0.208 mmol) in acetonitrile (1 mL) was added portionwise to a stirred solution of copper (II) bromide (0.25 mmol) and t-butylnitrite (0.31 mmol) in acetonitrile (2 mL) and the reaction mixture was maintained at 60 ° C. for 1 h. The reaction was diluted with ethyl acetate (15 mL) and washed twice with 1N HCl (aqueous). Purification was done by chromatography (silica, hexane; ethyl acetate) to yield the title compound (49%). ¹H NMR (CDCl₃) δ (ppm): 8.83 (s, 1H); 8.71 (s, 1H).

### EXAMPLE 3

### Preparation of Piperazine intermediates

GENERAL PROCEDURE A: Nucleophilic Displacement of Chloro-Heteroaryl at RT (Nitro Activating Group)

Piperazine (2-5 mmol) and 2-chloro-3-nitro-pyridine (1 mmol) were dissolved in DMF or acetonitrile (2-3 mL) and stirred for 5 min at RT. A slight exotherm was observed shortly after addition of the solvent. When TLC analysis showed that the reaction was complete, the mixture was diluted with DCM, and washed with water. The organic layer was dried, filtered and concentrated, then chromatography in 10% MeOH in DCM yielded the desired product.

| Example | Structure | Name | Yield |
|---|---|---|---|
| 3.1 | 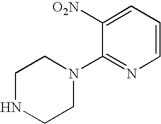 | 1-{3-nitropyridin-2-yl) piperazine | 63% |

| | |
|---|---|
| NMR | 3.00(m, 4H); 3.45(m, 4H); 6.75(dd, J=8.1, 4.5 Hz, 1H); 8.14 |
| | (dd, J=8.1, 1.8 Hz, 1H); 8.34(dd, J=4.5, 1.8 Hz, 1H) |

GENERAL PROCEDURE B: Amination of Heteroaryl Halides

i) BOC-Piperazine (2.4 mmol), 5-bromopyrimidine-4-carbonitrile (2 mmol), potassium carbonate (2.8 mmol), 2-dicyclohexylphosphino-2′,4′,6′triisopropyl-biphenyl (0.16 mmol), and tris(dibenzylideneacetone)dipalladium(0) (0.04 mmol) were dissolved in NMP (N-Methylpyrrolidinone) (5 mL) and stirred for 10 min at 200° C. The cooled mixture was diluted with ethyl acetate, and washed with water. The organic layer was dried, filtered and concentrated, then chromatography in 20-50% ethyl acetate in hexane yielded the desired BOC-protected intermediate. Note: The same procedure was used to prepare tert-butyl 4-(5-cyanopyrazin-2-yl)piperazine-1-carboxylate from 5-bromopyrazine-2-carbonitrile except that the reaction was carried out in DMF for 4 h. at 85° C.

| Example | Structure | Name | Yield |
|---|---|---|---|
| 3.2 | 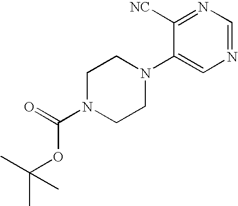 | tert-butyl 4-(4-cyanopyrimidin-5- yl)piperazine-1-carboxylate | 29% |
| NMR | 8.86(s, 1H); 8.59(s, 1H); 3.64(m, 4H); 3.37(m, 4H); 1.46(s, 9H) | | |
| 3.3 | 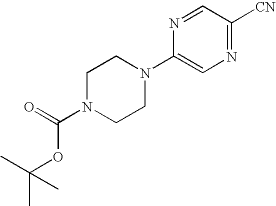 | tert-butyl 4-(5-cyanopyrazin-2- yl)piperazine-1-carboxylate | 88% |

| | |
|---|---|
| NMR | 8.35(s, 1H); 8.13(s, 1H); 3.75(m, 4H); 3.58(m, 4H); 1.49(s, 9H) |

ii) Removal of the BOC protecting group was accomplished under standard conditions (50% TFA/DCM) just prior to reaction with mesylate.

### EXAMPLE 4

### 5-5chloro-2-fluorophenyl)isoxazole-3-carbaldehyde

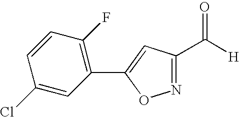

### (i) ethyl chloro(hydroxyimino)acetate

Concentrated hydrochloric acid (5.9 ml, 71.65 mmol) was added in a drop-wise manner to a solution of glycine ethyl ester hydrochloride (10 g, 71.65 mmol) in water (15 ml) at 0° C. Sodium nitrite (4.94 g, 71.65 mmol) in water (7.5 ml) was then added in a drop-wise manner to the resulting mixture, keeping the temperature below 5° C. After 10 min., the second equivalent of hydrochloric acid (5.9 ml, 71.65 mmol) as added drop-wise, followed by sodium nitrite (4.94 g, 71.65 mmol) in water (7.5 ml), again keeping the temperature below 5° C. The reaction mixture was stirred at 0° C. for 45 min., and then washed with ether. The organic phase was dried over anhydrous sodium sulfate and concentrated in vacuo to yield a yellow solid. The solid was recrystallized from hexanes, filtered and washed with hexanes to isolate a white crystalline solid (5.4153 g, 49.9%). ¹H NMR (300 MHz, CDCl₃): δ (ppm) 9.01 (s, 1H); 4.42(q, 2H); 1.41 (t, 3H).

### (ii) 4-chloro-2-ethynyl-1-fluorobenzene

A solution of 2-bromo-4-chloro-1-fluoro-benzene (2.91 ml, 23.9 mmol), ethynyl-trimethylsilane (5.2 ml, 36.5 mmol), palladium (II) acetate (108 mg, 0.478 mmol), and triphenyl-phosphine (250 mg, 0.965 mmol) in triethylamine (30 ml) was stirred at reflux overnight at 100° C. When the reaction was complete by GC/MS, the mixture was diluted with ethyl acetate and filtered through Celite®. The filtrate was concentrated in vacuo and the residue was absorbed on silica gel. The product was eluted using hexanes. Concentration in vacuo gave a brown oil in quantitative yield, which was used in the next step without further purification. ¹H NMR (300 MHz, CDCl₃): δ (ppm) 7.45 (m, 1H); 7.28 (m, 1H); 7.02 (t, 1H); 0.281 (s, 9H).

A mixture of (5-chloro-2-fluoro-phenylethynyl)-trimethylsilane (5.4196 g expected, 23.9 mmol) and potassium carbonate (16.50 g, 138.21 mmol) in MeOH (60 ml) was stirred at RT for 1 h. The reaction mixture was checked for completion using GC/MS, then diluted with hexanes and washed with water. The aqueous phase was extracted with hexanes (2×). The combined organic phase was washed with brine, dried over anhydrous sodium sulfate, filtered. Concentrated in vacuo gave the title compound (brown oil, quantitative yield, 3.74 g). ¹H NMR (300 MHz, CDCl₃): δ (ppm) 7.47 (m, 1H); 7.30 (m, 1H); 7.05 (t, 1H); 3.63 (s, 1H).

### (iii) ethyl 5-(5-chloro-2-fluorophenyl)isoxazole-3-carboxylate

Ethyl chloro(hydroxyimino)acetate (3.9271 g, 25.9 mmol) and sodium bicarbonate (7.00 g, 84.1 mmol) were added to a solution of 4-chloro-2-ethynyl-1-fluorobenzene (2.0019 g, 12.9 mmol) in toluene (50 ml). The reaction mixture was stirred at RT overnight, then filtered and the filtrate was concentrated in vacuo. The residue was taken up in ethyl acetate and washed with water. The organic phase was washed with brine, dried over sodium sulfate and concentrated in vacuo. Chromatography (silica gel, 0-2% acetoneahexanes) gave a yellow solid (1.4794 g, 42.5%). ¹H NMR (300 MHz, CDCl₃): δ (ppm) 8.00 (m, 1H); 7.44 (m, 1H); 7.19 (m, 2H); 4.50 (q, 2H); 1.45 (t, 3H).

### (iv) [5-(5-chloro-2-fluorophenyl)isoxazol-3-yl]methanol

Lithium aluminum hydride (95%, 0.2082 g, 5.486 mmol) was slowly added to a solution of ethyl 5-(5-chloro-2-fluorophenyl)isoxazole-3-carboxylate (1.4794 g, 5.486 mmol) in THF (20 ml). The reaction mixture was stirred at RT for 1 h. Sodium sulfate decahydrate was added to quench and the mixture was stirred at 63° C. for 15 min., and filtered through a Celite® pad using DCM. The filtrate was concentrated in vacuo to give a brown solid (600 mg, 48%, used without further purification). ¹H NMR (300 MHz, CDCl₃): δ (ppm) 7.96 (m, 1H); 7.40 (m, 1H); 7.17 (t, 1H); 6.83 (s, 1H); 4.86 (d, 2H).

### (v) 5-(5-chloro-2-fluorophenyl)isoxazole-3-carbaldehyde

A solution of [5-5-chloro-2-fluorophenyl)isoxazol-3-yl]methanol (600 mg, 2.636 mmol) in DCM was added drop-wise to a solution of pyridinium chlorochromate (852.32 mg, 3.953 mmol) in DCM (20 ml). The reaction mixture was stirred at RT overnight and filtered through silica, and the filtrate concentrated in vacuo. Chromatography (silica gel, ethyl acetate/hexanes (0-10%) gave a white solid (310 mg, 52.1%). ¹H NMR (300 MHz, CDCl₃): δ (ppm) 10.24 (s, 1H); 8.05 (m, 1H); 7.43 (m, I1H); 7.07 (m, 2H).

### EXAMPLE 5

### 1-[5-3-chlorophenyl)isoxazol-3-yl]ethanol

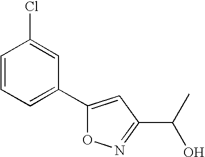

### (i) ethyl 4-(3-chlorophenyl)-2,4-dioxobutanoate

Sodium hydride (60% oil dispersion, 1.24 g, 31.1 mmol) was added in portions to a solution of 3-chloroacetophenone (4.0 g, 25.9 mmol) and diethyl oxalate (4.54 g, 31.1 mmol) in DMF (32 mL) at 0° C. The mixture stirred at RT for 1 h. and was then heated at 80° C. for 30 min. After cooling, the mixture was treated with 3N HCl and then diluted with ethyl acetate. The organic layer was washed with water (3×) and saturated brine, dried over anhydrous sodium sulfate, filtered and concentrated. Chromatography (silica, 0-10% ethyl acetate in hexanes) afforded the title compound (4.43 g, 67%, yellow solid). 1H NMR (CDCl₃) δ (ppm): 15.12 (br s, 1H), 7.98 (s, 1H), 7.88 (d, 1H), 7.58 (d, 1H), 7.47 (t, 1H), 7.05 (s, 1H), 4.39 (m, 2H), 1.41 (m, 3H).

### (ii) ethyl 5-(3-chlorophenyl)isoxazole-3-carboxylate

A solution of ethyl 4-(3-chlorophenyl)-2,4-dioxobutanoate (3.0 g, 11.8 mmol) and hydroxylamine hydrochloride (2.46 g, 35.4 mmol) in MeOH (60 mL) was heated at 80° C. for 4 h. After cooling, the mixture was filtered and washed with cold MeOH to afford 5-(3-chloro-phenyl)-isoxazole-3-carboxylic acid ethyl ester (2.0 g, 71%, white solid). 1H NMR (CDCl₃) δ (ppm): 7.82 (s, 1H), 7.72 (m, 1H), 7.47 (m, 2H), 4.03 (s, 3H). Mixture of both methyl and ethyl ester (mostly methyl).

### (iii) 1-[5-(3-chlorophenyl)isoxazol-3-yl]ethanone

A solution of ethyl 5-(3-chlorophenyl)isoxazole-3-carboxylate (300 mg, 1.19 mmol) in toluene (5 ml) was added to a mixture of methyl magnesium iodide (3M in diethyl ether) (0.79 ml, 2.38 mmol), toluene (1 ml), tetrahydrofuran (0.39 ml, 4.77 mmol) and triethylamine (1 ml, 7.15 mmol) at 0° C. The resulting mixture was stirred at 0° C. for 5 h, then quenched with 1N hydrochloric acid (aqueous, 6.5 ml, 6.5 mmol), diluted with toluene (35 ml), sequentially washed with water (50 ml), saturated sodium bicarbonate (aqueous, 30 ml), water (50 ml) and brine (30 ml). The organic phase was concentrated in vacuo. The isolated residue was dissolved in MeOH (8 ml) and 20% potassium hydroxide (aqueous, 1 ml). The mixture was stirred at 45° C. for 30 min. and concentrated in vacuo. The residue was dissolved in toluene (60 ml), sequentially washed with water (50 ml), saturated sodium bicarbonate (aqueous, 50 ml) and water (50 ml). The organic phase was concentrated in vacuo. Chromatography (silica gel, 2% ethyl acetate in hexanes) gave the title compound (white solid, 156 mg, 60%). ¹H-NMR (CDCl₃), δ (ppm): 7.77 (m, 1H), 7.66 (m, 1H), 7.42 (m, 2H), 6.90 (s, 1H), 2.69 (s, 3H).

### (iv) 1-[5-(3-chlorophenyl)isoxazol-3-yl]ethanol

A mixture of 1-[5-(3-chlorophenyl)isoxazol-3-yl]ethanone (100 mg, 0.45 mmol), sodium borohydride (34 mg, 0.90 mmol) and MeOH (3 ml) was stirred at RT for 3 h. The reaction was quenched with water (30 ml) and brine (30 ml), and the product was extracted with DCM (3×30 ml). The organic layer was dried (sodium sulfate), filtered and concentrated in vacuo to give the title compound (white solid, 110 mg). ¹H-NMR (CDCl₃), δ (ppm): 7.69 (m, 1H), 7.59 (m, 1H), 7.37 (m, 2H), 6.59 (s, 1H), 5.07 (q, 1H), 3.45 (bs, 1H), 1.58 (d, 3H)

### EXAMPLE 6

### 1-[5-(5-chloro-2-fluorophenyl)isoxazol-yl]ethanol

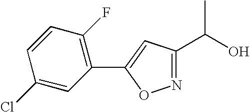

Methylmagnesium iodide (3M diethyl ether) (0.766 ml, 2.298 mmol) was added dropwise to a solution of 5-(5-chloro-2-fluorophenyl)isoxazole-3-carbaldehyde (259.3 mg, 1.149 mmol) in THF (5 ml) at 0° C. The mixture was stirred at 0° C. for 1.5 h., then ethyl acetate and ammonium chloride were added. The organic phase was isolated, washed with brine, dried over anhydrous sodium sulfate and concentrated in vacuo. Chromatography (silica gel, 0-20% ethyl acetate/hexanes) gave the title compound (clear oil, 190 mg, 68.3%). ¹H NMR (300 MHz, CDCl₃): δ (ppm) 7.95 (m, 1H); 7.40 (m, 1H); 7.17 (t, 1H); 6.80 (s, 1H); 5.12 (m, 1H); 2.22 (d, 1H); 1.64 (d, 3H).

### EXAMPLE 7

### 3-[3-(1-hydroxyethyl)isoxazol-5yl]benzonitrile

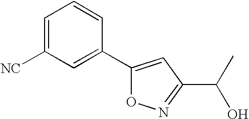

### (i) methyl 5-(3-iodophenyl)isoxazole-3-carboxylate

Sodium hydride (60% oil dispersion, 4.9 g, 122.8 mmol) was added in portions to a solution of 3-iodoacetophenone (25.18 g, 102.3 mmol) and dimethyl oxalate (14.5 g, 122.8 mmol) in DMF (125 mL) at 0° C. The mixture stirred at RT for 1 h. and was then heated at 115° C. for 1 h. After cooling, the mixture was treated with 3N HCl and then diluted with ethyl acetate. The organic layer was washed with water (3×) and saturated brine, dried over anhydrous sodium sulfate, filtered and concentrated. Chromatography (silica, 0-10% ethyl acetate in hexanes) afforded the intermediate (24.21 g, 71.3%, yellow solid).

A solution of the intermediate (33.87 g, 102 mmol) and hydroxylamine hydrochloride (21.3 g, 306 mmol) in MeOH (450 mL) was heated at reflux for 4 h. After cooling, the mixture was filtered and washed with cold MeOH to afford the title compound (24.10 g, 72%, brown solid). 1H NMR (CDCl₃) δ (ppm): 8.18 (m, 1H), 7.82 (t, 2H), 7.26 (t, 1H), 6.97 (s, 1H), 4.03 (s, 3H).

### (ii) [5-(3-iodophenyl)isoxazol-3-yl]methanol

DIBAL (55.8 mL, 1.5M in toluene, 83.7 mmol) was slowly added to a solution of methyl 5-(3-iodophenyl)isoxazole-3-carboxylate (12 g, 36.5 mmol) in toluene (60 ml) and THF (60 mL) at −78° C. The resulting mixture was stirred at −78° C. overnight, then allowed to warm slowly to RT. The reaction was quenched with a mixture of ice and saturated ammonium chloride (aqueous). The product was extracted with ethyl acetate, and the organic layer was washed with brine, dried over sodium sulfate and concentrated in vacuo to give the title compound (off-white solid, 10.5 g, 95.6%). ¹H-NMR (CDCl₃), δ (ppm): 8.12 (m, 1H), 7.76 (ddm, 2H), 7.21 (t, 1H), 6.62 (s, 1H), 4.83 (s, 2H), 2.45 (br s, 1H).

### (iii) 5-(3-iodophenyl)isoxazole-3-carbaldehyde

A mixture of [5-(3-iodophenyl)isoxazol-3-yl]methanol (8.5 g, 28.23 mmol) and pyridinium chlorochromate (9.13 g, 42.35 mmol) in DCM (150 ml) was stirred at RT overnight The mixture was diluted with 15% ethyl acetate in hexanes and passed thorough a short plug of silica gel, eluting with additional 15% ethyl acetate in hexanes. The eluent was concentrated in vacuo to give the title compound (pale yellow solid, 7.0 g, 83%). ¹H-NMR (CDCl₃), δ (ppm): 10.21 (s, 1H), 8.19 (m, 1H), 7.83 (ddm, 2H), 7.27 (m, 1H), 6.93 (s, 1H).

### (iv) 3-[3-(1-hydroxyethyl)isoxazol-5-yl]benzonitrile

Methyl magnesium iodide (33 mL, 3M in diethyl ether, 99 mmol) was added to a cold (0° C.) solution of 5-(3-iodophenyl)isoxazole-3-carbaldehyde (7.5 g, 25 mmol) in THF (100 mL). The reaction mixture was stirred at 0° C. for 1 h and quenched with saturated ammonium chloride. The product was extracted with ethyl acetate, and the organic layer was washed with brine, dried over a mixture of sodium sulfate and silica gel. The filtrate was concentrated in vacuo and chromatography (silica, 15-50% ethyl acetate in hexanes) gave the crude iodo-isoxazole-alcohol (pale yellow oil, 6.5 g, contaminated with ˜33% 1-(5-phenylisoxazol-3-yl)ethanol).

Tert-butyldimethylchlorosilane (2.5 g, 2.3 mmol) was added to a solution of crude 1-[5-(3-iodophenyl)isoxazol-3-yl]ethanol (4.9 g, 15.55 mmol) and DBU (2.53 g, 2.13 mmol) in DCM (60 mL) and the reaction was stirred at RT for 3 h. Tert-butyldimethylchlorosilane (2.5 g, 2.3 mmol) and DBU (2.53 g, 2.13 mmol) were added and stirring was continued for 15 min until TLC indicated the alcohol was consumed. The product was partitioned between saturated ammonium chloride and DCM, and the organic layer was dried and concentrated in vacuo to give the iodo-isoxazole-silyl ether (pale yellow solid, 8.4 g crude).

A mixture of the crude silyl ether, zinc cyanide (1.6 g, 13.69 mmol), tetrakis(triphenylphosphine)palladium(0) (1.58 g, 1.37 mmol) in DMF (100 mL) was stirred at 82° C. for 10 min. The mixture was diluted with ethyl acetate and filtered through Celite®. The filtrate was concentrated in vacuo and diluted with DCM. The solution was washed with water, dried over sodium sulfate and filtered. Chromatography (preadsorbed on silica, 1-5% ethyl acetate in hexane) gave the pure cyano-isoxazole-silyl ether (off-white solid, 3.83 g, 46.5% over 3 steps). ¹H-NMR (CDCl₃), δ (ppm): 8.07 (m, 1H), 8.04 (dm, 1H), 7.73 (dm, 1H), 7.62 (t, 1H), 6.66 (s, 1H), 5.09 (q, 1H), 1.54 (d, 3H), 0.93 (s, 9H), 0.13 (s, 3H), 0.06 (s, 3H).

TBAF (20 mL, 1M in THF, 20 mmol) was added to a solution of the pure cyano-isoxazole-silyl ether (3.83 g, 11.66 mmol) in THF (40 mL) at 0° C. and the mixture was stirred overnight at RT. The product was partitioned between DCM and water. The organic layer was washed with brine and dried over magnesium sulfate. Silica gel was added and the mixture was passed through a plug of silica gel using 50% ethyl acetate in hexanes. The eluent was concentrated in vacuo and the residue was triturated with hexanes to give the title compound (off-white solid, 2.5 g, 100%). ¹H-NMR (CDCl₃), δ (ppm): 8.07 (m, 1H), 8.03 (dm, 1H), 7.75 (dm, 1H), 7.62 (t, 1H), 6.7 (s, 1H), 5.13 (q, 1H), 1.64 (d, 3H)

### EXAMPLE 8

### 5-(3-chlorophenyl)isoxazole-3-carboxylic acid

### i) [5-(3-chlorophenyl)isoxazol-3-yl]methanol

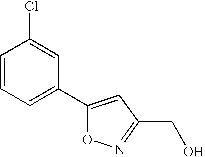

Lithium aluminum hydride (320 mg, 8.4 mmol) was slowly added to a solution of ethyl 5-(3-chlorophenyl)isoxazole-3-carboxylate (2.0 g, 8.4) in THF (100 ml) at RT. After 1 h, the reaction mixture was quenched with water and then extracted with ethyl acetate. The organic layer was washed with water and saturated brine, dried over anhydrous sodium sulfate, filtered, and concentrated. The resulting residue was then purified by flash column chromatography using 15-40% ethyl acetate in hexane to afford the title compound (1.32 g, 75%, yellow solid). ¹H NMR (CDCl₃) δ (ppm): 7.78 (s, 1H), 7.68 (m, 1H), 7.43 (m, 2H), 6.63 (s, 1H), 4.84 (d, 2H), 2.23 (t, 1H).

### ii) 5-(3-chlorophenyl)isoxazole-3-carboxylic acid

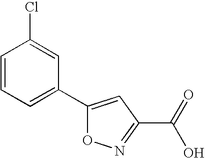

Potassium permanganate (4.1 g, 26.23 mmol) was added to a cooled (10° C.) solution of [5-(3-chlorophenyl)isoxazol-3-yl]methanol (1.1 g, 5.25 mmol) in acetone (20 mL). After 2 h, the reaction mixture was filtered through Celite®, rinsing with acetone and water. The acetone was removed in vacuo and the aqueous layer was acidified with 1N HCl(aq). The product was extracted with ethyl acetate (2×) and the organic layer was dried, filtered, and concentrated in vacuo. Trituration with hexanes afforded the title compound (286 mg, 24%, off-white solid).

### EXAMPLE 9

### Preparation of 1,3-oxazole intermediate-2-(3-chlorophenyl)-1,3-oxazole-4-carboxylic acid

### i) Methyl 2-[(3chlorobenzoyl)amino]-3-hydroxypropanoate

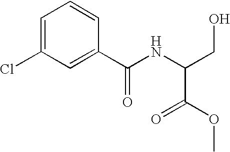

N-methylmorpholine (7.0 ml, 63.8 mmol) and EDCI (4.97 g, 31.9 mmol) were added to a mixture of 3-chlorobenzoic acid (5.0 g, 31.9 mmol), serine methyl ester hydrochloride (6.1 g, 31.9 mmol) and HOBt (4.31 g, 31.9 mmol) in DMF (100 ml) at 0° C. The mixture was allowed to warm to RT and stirred for 18 h. The mixture was diluted with ethyl acetate (300 ml) and then washed with water (3×250 ml) followed by brine. The organic extract was dried over Na₂SO₄ (anhydrous) and then concentrated in vacuo to give the title compound (7.2 g, 93%, pale yellow solid). ¹H NMR (CDCl₃) δ (ppm): 7.78 (s, 1 H), 7.66 (d, 1 H), 7.45, (dd, 1 H), 7.34 (t, 1 H), 7.25 (br, d, 1H), 4.82 (m, 1 H), 4.08 (m, 2 H), 3.79 (s, 3 H), 3.19 (br, t, 1H).

### ii) Methyl 2-(3-chlorophenyl)-1,3-oxazole-4-carboxylate

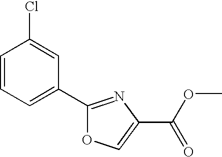

Deoxo-fluor®/bis(2-methoxyethyl)amino-sulfur trifluoride (7.2 g, 32.6 mmol) was added dropwise to a solution of methyl 2-[(3-chlorobenzoyl)amino]-3-hydroxypropanoate (7.2 g, 29.6 mmol) in DCM at −20° C. After stirring at this temperature for 30 min., BrCCl₃ (3.6 g, 18.1 mmol) was added dropwise followed by DBU (2.79 g, 18.1 mmol). The mixture was then stirred at 2-3° C. for 8 h ad then quenched with saturated NaHCO_{3(aq)} followed by extraction with ethyl acetate. The organic extract as then washed with brine and dried over Na₂SO₄ (anhydrous). Purification was performed by flash column chromatography on silica gel using ethyl acetate in hexanes as eluent to afford methyl 2-(3-chlorophenyl)-1,3-oxazole-4-carboxylate (4.1 g, 59%, yellow solid). ¹H NMR (CDCl₃) δ (ppm): 8.30 (s, 1 H), 8.12 (d, 1 H), 7.98 (dd, 1 H), 7.45 (m, 2 H), 3.96 (s, 3 H).

### iii) 2-(3-chlorophenyl)-1,3-oxazole-4-carboxylic acid

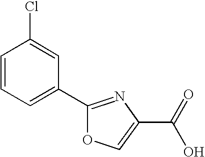

Sodium hydroxide (10 mL, 1M, 10 mmol) was added to a suspension of methyl 2-(3-chlorophenyl)-1,3-oxazole-4-carboxylate (1.0 g, 54.21 mmol) in MeOH (20 mL). The resulting mixture was heated at 60° C. for 15 min, then diluted with a mixture of ice and water. The resulting mixture was acidified with 1N HCl(aq) until pH 3. The solid product was collected by filtration, rinsed with water and dried under vacuum to afford the title compound (789 mg, 84%).

### EXAMPLE 10

### 1-[5-(3-chlorophenyl)-1,2,4-oxadiazol-3-yl]ethanol

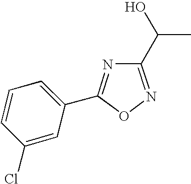

### (i) N′,2-dihydroxypropanimidamide

A solution of sodium hydroxide (3.09 g, 77.37 mmol) and hydroxylamine hydrochloride (5.38 g, 77.37 mmol) in ethanol (40 ml) was stirred at RT for 30 min. The solution was filtered and the filtrate was slowly added to 2-hydroxy-propionitrile (5.05 ml, 70.34 mmol). The mixture was left to stir at RT overnight, then concentrated to yield the title compound (white solid, 6.3728 g, 87%). ¹H NMR (300 MHz, DMSO): δ (ppm) 8.91 (s, 1H); 5.23 (s, 2H); 5.11 (s, 1H); 4.01 (q, 1H); 1.21 (d, 3H).

### (ii) 1-[5-(3-chlorophenyl)-1,2,4-oxadiazol-3-yl]ethanol

3-Chloro-benzoyl chloride (2.71 ml, 21.13 mmol) was added to a solution of 2,N-dihydroxy-propionamidine (2.0 g, 19.21 mmol) in pyridine (25 mL) at 0° C. The reaction mixture was stirred for 2.5 h. while allowing it to warm to RT, then heated at 140° C. for 1 h (sealed vial). The reaction mixture was poured into ice water and extracted with DCM (×2). The organic layer was washed with brine, dried over anhydrous sodium sulfate and concentrated in vacuo. The resulting brown solid was recrystallized from 10% ethyl acetate in hexanes to yield the title compound (light brown solid, 2.1828 g, 46.8%). ¹H NMR (300 MHz, CDCl₃): δ (ppm) 8.16 (m, 1H); 8.04 (m, 1H); 7.58 (m, 1H); 7.50 (m, 1H); 5.12 (q, 1H); 2.71 (s, 1H); 1.70 (d, 3H).

### EXAMPLE 11

### 1-[3-(3-chlorophenyl)-1,2,4oxadiazol-5-yl]ethanol

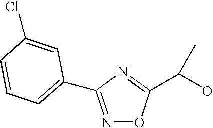

### (i) 1-[3-(3-chlorophenyl)-1,2,4-oxadiazol-5-yl]ethyl acetate

A few drops of DMF was added to a mixture of 2-acetoxypropionic acid (540 mg, 4.1 mmol) and oxalyl chloride (4 mL, 2M in DCM, 8 mmol) in DCM (4 mL) at 0° C. and bubbling was observed. The mixture was stirred at 0° C. for 30 min and then warmed to RT for 1.5 h. Toluene (5 mL) was added to ensure removal of excess oxalyl chloride during concentration in vacuo. 3-Chloro-N′-hydroxybenzenecarboximidamide (599 mg, 3.51 mmol) was added to a solution of the acid chloride in ethyl acetate (30 mL). A saturated aqueous solution of sodium bicarbonate was added and the reaction mixture was stirred vigorously for 30 min. The layers were separated and the organic layer was washed with brine, dried over sodium sulfate and concentrated in vacuo. DMF (5 mL) was added to the residue and the resulting mixture was stirred for 1.5 h. at 135° C. The solvent was removed in vacuo and chromatography (product preadsorbed on silica, 5-10% ethyl acetate in hexane) yielded the product (452 mg, 48.3%). ¹H NMR (300 MHz, CDCl₃): δ (ppm) 8.11 (m, 1H), 7.99 (dm, 1H), 7.51 (dm, 1H), 7.46 (t, 1H), 6.11 (q, 1H), 2.21 (s, 3H), 1.77 (d, 3H).

### (ii) 1-[3-(3chlorophenyl)-1,2,4-oxadiazol-5-yl]ethanol

Lithium hydroxide (3.7 mL, 0.5M aqueous, 1.85 mmol) was added to a solution of 1-[3-(3-chlorophenyl)-1,2,4-oxadiazol-5-yl]ethyl acetate (451.6 mg, 1.69 mmol) in THF (6 mL) and MeOH (2.5 mL). The mixture was stirred for 2 h, then partitioned between ethyl acetate and water. The organic layer was washed with brine, dried over sodium sulfate and the solvent was removed in vacuo. Chromatography (silica, 15-20% ethyl acetate in hexanes) gave the title compound (white solid, 382.9 mg, 100%). ¹H NMR (300 MHz, CDCl₃): δ (ppm) 8.11 (m, 1H), 7.99 (dm, 1H), 7.51 (dd, 1H), 7.47 (t, 1H), 5.19 (m, 1H), 2.73 (d, 1H), 1.75 (d, 3H).

### EXAMPLE 12

### General Procedure: Triazole Ring Formation from Acetylene

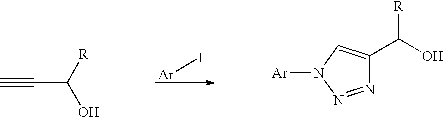

A mixture of aryl iodide or bromide (1 mmol), propargyl alcohol (1 mmol), L-proline (0.2 mmol), sodium carbonate (00.2 mmol), sodium azide (1.2 mmol), sodium ascorbate (0.1 mmol) and copper sulfate pentahydrate (0.05 mmol) in 2 ml of 9:1 DMSO:H₂O was stirred overnight at 65° C. The mixture was diluted with ethyl acetate and washed sequentially with water and dilute ammonium hydroxide (3×). Purification by SPE column chromatography (silica, 7-10% MeOH in DCM) gave the triazole. Reference: Organic Letters. 2004, Vol. 6, No. 22, 3897-3899.

The following compounds were prepared in this manner:

| Example | Structure | Name | Yield |
|---|---|---|---|
| 12.1 | 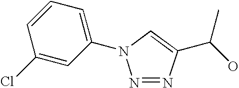 | 1-[1-(3-chlorophenyl)-1H-1,2,3- triazol-4-yl]ethanol | beige-brown solid, 951.5 mg, 60% |
| NMR | 7.95(s, 1H); 7.80(m, 1H); 7.64(m, 2H); 7.45(m, 2H); 5.2(m, 1H); 2.0(d, 1H); | | |
| | 1.68(d, 3H) | | |
| 12.2 | 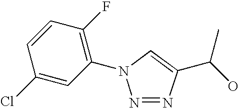 | 1-[1-(5-chloro-2-fluorophenyl)- 1H-1,2,3-triazol-4-yl]ethanol | off white solid, 59.9 mg, 4% |

| | |
|---|---|
| NMR | 8.03(m, 2H); 7.39(m, 1H); 7.27(m, 1H); 5.20(q, 1H); 2.85(s, 1H); 1.69(d, 3H) |

### EXAMPLE 13

### 5-(1-chloroethyl)-3-(3-chlorophenyl)-1,2,4-oxadiazole

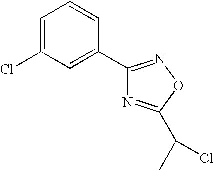

### (i) 3-chloro-N′-hydroxybenzenecarboximidamide

Sodium hydroxide (8.2 g in 50 mL water) and hydroxylamine hydrochloride (16 g in 20 mL water) were added to a solution of 3-chloro-benzonitrile (28 g, 203.5 mmol) at 80° C. in ethanol (50 mL). The resulting mixture was stirred for 2 h. at 80° C. The solvent was removed in vacuo. to afford the title compound (29.82 g, 85.9%). ¹H NMR (300 MHz, CDCl₃): δ (ppm) 7.65 (s, 1H), 7.52 (d, 1H), 7.41 (d, 1H), 7.35 (t, 1H), 4.86 (br, 2H), 1.68 (br, 1H).

### (ii) 5-(1-chloroethyl)-3-(3-chlorophenyl)-1,2,4-oxadiazole

2-Chloropropanoyl chloride (8.94 g, 70.4 mmol) was added in a drop-wise manner to a solution of 3-chloro-N′-hydroxybenzenecarboximidamide (10.0 g, 58.7 mmol) in ethyl acetate (200 mL) at 10° C. (ice bath). A saturated aqueous solution of sodium bicarbonate was added and the reaction mixture was stirred vigorously for 10 min. The layers were separated and the organic layer was washed sequentially with water and brine, dried over sodium sulfate and concentrated in vacuo. DMF (60 mL) was added to the residue and the resulting mixture was stirred for 1.5 h. at 135° C. The mixture was diluted with water and DCM, and the layers were separated. The organic layer was washed with water, and brine and dried with sodium sulfate, filtered, and concentrated in vacuo. Chromatography (product preadsorbed on silica, 5% ethyl acetate in hexane) yielded the product (7.5 g, 52.6%). ¹H NMR (300 MHz, CDCl₃): δ (ppm) 8.11 (s, 1H), 7.99 (d, 1H), 7.52 (d, 1H), 7.45 (t, 1H), 5.28 (q, 1H), 2.05 (d, 3H).

### EXAMPLE 14

### General Procedure: Mesylation of Alcohol

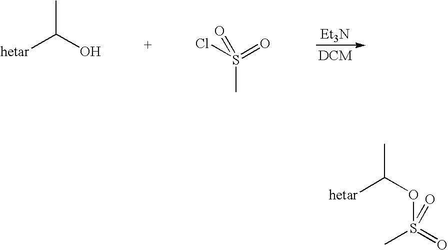

Methanesulfonyl chloride (1.5 mmol) and triethylamine (2 mmol) were added to a solution of heteroaryl alcohol (1 mmol) in DCM (10-15 ml) at 0° C. The reaction mixture was stirred at 0° C. for 30 min., then washed with cold saturated sodium bicarbonate. The organic layer was washed with brine, dried with sodium sulfate and concentrated in vacuo to give the title compound which was used without further purification.

The following mesylates were synthesized using the procedure above.

| Example | Structure | Name | Yield |
|---|---|---|---|
| 14.1 | 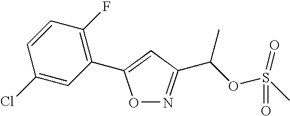 | 1-[5-(5-chloro-2-fluorophenyl) isoxazol-3-yl]ethyl methane sulfonate | White solid, 249.1 mg, 98.7% |
| NMR | 7.95(m, 1H); 7.43(m, 1H); 7.20(t, 1H); 6.86(s, 1H); 6.95(q, 1H); 3.05(s, 3H); 1.85 | | |
| | (d, 3H) | | |
| 14.2 | 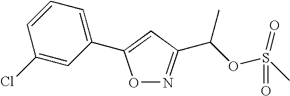 | 1-[5-(3-Chlorophenyl)isoxazol- 3-yl]ethyl methanesulfonate | Orange oil, 622.7 mg, 92.5% |
| NMR | 7.79(m, 1H); 7.69(m, 1H); 7.45(m, 2H); 6.69(s, 1H); 5.92(q, 1H); 3.06(s, 3H); 1.84 | | |
| | (d, 3H) | | |
| 14.3 | 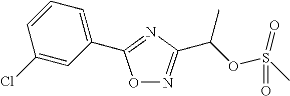 | 1-[5-(3-chlorophenyl)-1,2,4- oxadiazol-3-yl]ethyl methane sulfonate | Brown solid, 552.4 mg, 51.2% |
| NMR | 8.05(m, 1H); 7.97(m, 1H); 7.55(m, 1H); 7.46(m, 1H); 5.89(q, 1H); 3.14(s, 3H); | | |
| | 1.85(d, 3H) | | |
| 14.4 | 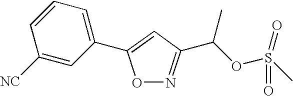 | 1-[5-(3-cyanophenyl)isoxazol-3- yl]ethyl methanesulfonate | off-white solid, 3.65 g, 100% |
| NMR | 8.09(m, 1H), 8.04(dm, 1H), 7.77(dm, 1H), 7.65(t, 1H), 6.77(s, 1H), 5.94(q, 1H), | | |
| | 3.08(s, 3H), 1.85(d, 3H) | | |
| 14.5 | 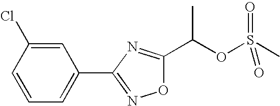 | 1-[3-(3-chlorophenyl)-1,2,4- oxadiazol-5-yl]ethyl methane sulfonate | white oil, 134.8 mg, 100% |
| NMR | 8.05(m, 1H); 7.96(m, 1H); 7.46(m, 2H); 5.98(q, 1H); 3.20(s, 3H); 1.93(d, 3H) | | |
| 14.6 | 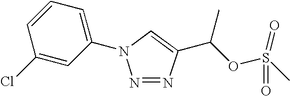 | 1-[1-(3-chlorophenyl)-1H-1,2,3- triazol-4-yl]ethyl methane sulfonate | brown oil, 1.23 g, 96% |
| NMR | 8.10(s, 1H); 7.81(s, 1H); 7.67(m, 1H); 7.46(m, 2H); 6.00(q, 1H); 3.07(s, 3H); 1.79 | | |
| | (d, 3H) | | |
| 14.7 | 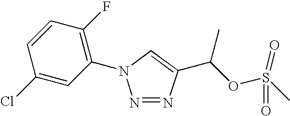 | 1-[1-(5-chloro-2-fluorophenyl)- 1H-1,2,3-triazol-4-yl]ethyl methane sulfonate | Yellow- brown oil, 72.1 mg, 91% |

| | |
|---|---|
| NMR | 8.20(m, 1H); 8.04(m, 1H); 7.44(m, 1H); 7.30(m, 1H); 6.05(q, 1h); 3.70(s, 1H); |
| | 3.07(s, 3H); 1.80(d, 3H) |

### EXAMPLE 15

### General Procedure: Piperazine Displacement of Mesylate

A mixture of the appropriate mesylate (1 mmol), aryl piperazine (1.5 mmol) and potassium carbonate (2 mmol) in acetonitrile (15 ml) was stirred at 80° C. overnight. The reaction mixture was diluted with ethyl acetate and water. The organic layer was washed with saturated sodium bicarbonate and brine, dried over anhydrous sodium sulfate, and concentrated in vacuo. SPE column chromatography (silica gel, 0-70% ethyl acetate in hexanes) yielded the desired compound.

The following compounds were synthesized using the above procedure.

| Example | Structure | Name | Yield |
|---|---|---|---|
| 15.1 | 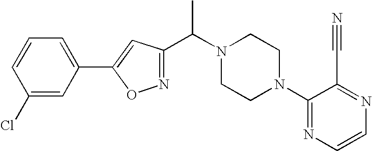 | 3-(4-{1-[5-(3-chlorophenyl)isoxazol- 3-yl]ethyl}piperazin-1-yl)pyrazine-2- carbonitrile | Yellow oil, 29.5 mg, 28% |
| NMR | 8.24(m, 1H); 8.00(m, 1H); 7.78(m, 1H); 7.69(m, 1H); 7.42(m, 2H); 6.57(s, 1H); | | |
| | 3.93(q, 1H); 3.87(m, 4H); 2.70(m, 4H); 1.51(d, 3H) | | |
| 15.2 | 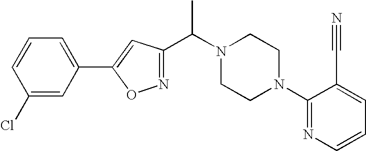 | 2-(4-{1-[5-(3-chlorophenyl)isoxazol- 3-yl]ethyl}piperazin-1-yl) nicotinonitrile | Yellow oil, 39.7 mg pure, 38% |
| NMR | 8.33(m, 1H); 7.78(m, 2H); 7.75(m, 1H); 7.42(m, 2H); 6.75(m, 1H); 6.58(s, 1H); | | |
| | 3.91(q, 1H); 3.76(m, 4H); 2.70(m, 4H); 1.51(d, 3H) | | |
| 15.3 | 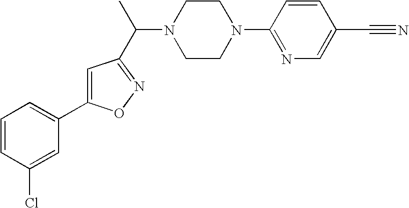 | 6-(4-{1-[5-(3-chlorophenyl)isoxazol- 3yl]ethyl}piperazin-1-yl) nicotinonitrile | Yellow oil, 42.4 mg pure, 41% |
| NMR | 8.40(m, 1H); 7.77(m, 1H); 7.61(m, 1H); 7.59(m, 1H); 7.42(m, 2H); 6.57(s, 1H); | | |
| | 3.91(q, 1H); 3.71(m, 4H); 2.64(m, 4H); 1.51(d, 3H) | | |
| 15.4 | 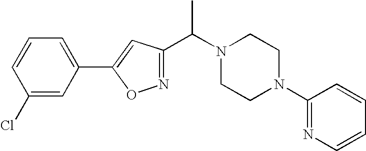 | 1-{1-[5-(3-chlorophenyl)isoxazol-3- yl]ethyl}-4-pyridin-2-ylpiperazine | Yellow oil, 65.0 mg, 66% |
| NMR | 8.19(m, 1H); 7.79(m, 1H); 6.68(m, 1H); 7.42(m, 1H); 7.41(m, 2H); 6.63(m, | | |
| | 3H); 3.89(q, 1H); 3.57(m, 4H); 2.67(m, 4H); 1.51(d, 3H) | | |
| 15.5 | 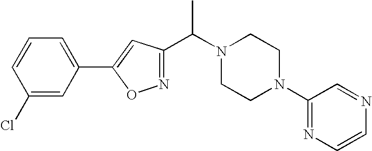 | 2-(4-{1-[5-(3-chlorophenyl)isoxazol- 3-yl]ethyl}piperazin-1-yl)pyrazine | orange solid, 28.9 mg, 29.5% |
| NMR | 8.13(m, 1H); 8.06(m, 1H); 7.86(m, 1H); 7.79(m, 1H); 7.68(m, 1H); 7.42(m, | | |
| | 3H); 6.59(s, 1H); 3.93(q, 1H); 3.63(m, 4H); 2.69(m, 4H); 1.52(d, 3H) | | |
| 15.6 | 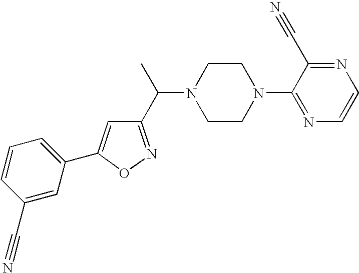 | 3-(4-{1-[5-(3-cyanophenyl)isoxazol- 3-yl]ethyl}piperazin-1-yl)pyrazine-2- carbonitrile | Yellow oil, 28.6 mg, pure, 28% |
| NMR | 8.25(m, 1H); 8.04(m, 3H); 7.73(m, 1H); 7.64(m, 1H); 6.65(s, 1H); 3.94(q, 1H); | | |
| | 3.86(m, 4H); 2.71(m, 4H); 1.52(d, 3H) | | |
| 15.7 | 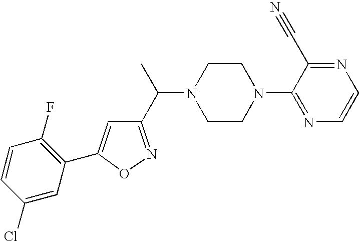 | 3-(4-{1-[5-(5-chloro-2-fluorophenyl) isoxazol-3-yl]ethyl}piperazin-1-yl) pyrazine-2-carbonitrile | Yellow oil, 12.3 mg, 19% |
| NMR | 8.25(m, 1H); 8.01(m, 1H); 7.95(m, 1H); 7.39(m, 1H); 7.17(t, 1H); 6.74(s, 1H); | | |
| | 3.96(q, 1H); 3.86(m, 4H); 2.71(m, 4H); 1.52(d, 3H) | | |
| 15.8 | 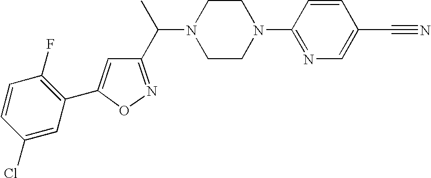 | 6-(4-{1-[5-(5-chloro-2-fluorophenyl) isoxazol-3-yl]ethyl}piperazin-1-yl) nicotinonitrile | Yellow oil, 24.9 mg, 39% |
| NMR | 8.39(m, 1H); 7.93(m, 1H); 7.60(m, 1H); 7.41(m, 1H); 7.17(t, 1H); 6.73(s, 1H); | | |
| | 6.58(m, 1H); 3.95(q, 1H); 3.72(m, 4H); 2.63(m, 4H); 1.52(d, 3H) | | |
| 15.9 | 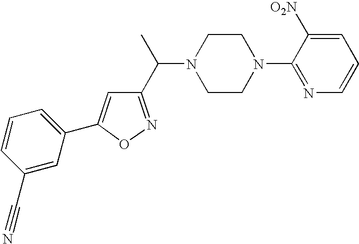 | 3-(3-{1-[4-(3-nitropyridin-2-yl) piperazin-1-yl]ethyl}isoxazol-5-yl) benzonitrile | Yellow oil, 47.7 mg, 69% |
| NMR | 8.33(m, 1H); 8.05(m, 3H); 7.73(m, 1H); 7.63(m, 1H); 6.75(m, 1H); 6.65(s, 1H); | | |
| | 3.93(q, 1H); 3.51(m, 4H); 2.66(m, 4H); 1.50(d, 3H) | | |
| 15.10 | 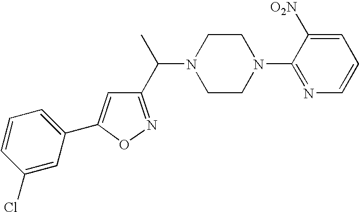 | 1-{1-[5-(3-chlorophenyl)isoxazol-3- yl]ethyl}-4-(3-nitropyridin-2-yl) piperazine | Yellow oil, 52.7 mg, 77% |
| NMR | 8.31(m, 1H); 8.13(m, 1H); 7.78(m, 1H); 7.69(m, 1H); 7.42(m, 2H); 6.75(m, | | |
| | 1H); 6.58(s, 1H); 3.92(q, 1H); 3.54(m, 4H); 2.66(m, 4H); 1.50(d, 3H) | | |
| 15.11 | 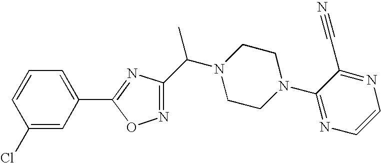 | 3-(4-{1-[5-(3-chlorophenyl)-1,2,4- oxadiazol-3-yl]ethyl}piperazin-1-yl) pyrazine-2-carbonitrile | orange oil, 93.2 mg, 71% |
| NMR | 8.31(m, 1H); 8.22(m, 1H); 8.05(m, 1H); 8.00(m, 1H); 7.59(m, 1H); 7.50(m, | | |
| | 1H); 4.10(q, 1H); 3.83(m, 4H); 2.77(m, 4H); 1.58(d, 3H) | | |
| 15.12 | 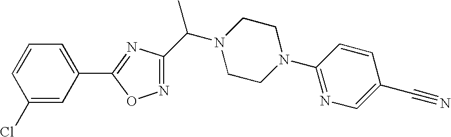 | 6-(4-{1-[5-(3-chlorophenyl)-1,2,4- oxadiazol-3-yl]ethyl}piperazin-1-yl) nicotinonitrile | Yellow oil, (37.6 mg, 34% |
| NMR | 8.39(m, 1H); 8.17(m, 1H); 8.05(m, 1H); 7.56(m, 3H); 6.58(m, 1H); 4.10(q, 1H); | | |
| | 3.74(m, 4H); 2.73(m, 4H); 1.60(d, 3H) | | |
| 15.13 | 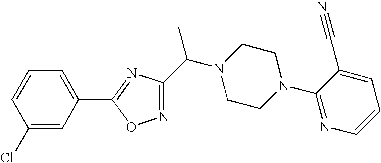 | 2-(4-{1-[5-(3-chlorophenyl)-1,2,4- oxadiazol-3-yl]ethyl}piperazin-1-yl) nicotinonitrile | Yellow oil, 18.2 mg, 19% |
| NMR | 8.33(m, 1H); 8.18(m, 1H); 8.05(m, 1H); 7.76(m, 1H); 7.57(m, 1H); 7.49(m, | | |
| | 1H); 6.75(m, 1H); 4.08(q, 1H); 3.79(m, 4H); 2.80(m, 4H); 1.60(d, 3H) | | |
| 15.14 | 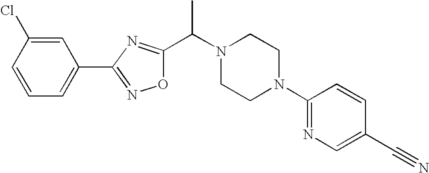 | 6-(4-{1-[3-(3-chlorophenyl)-1,2,4- oxadiazol-5-yl]ethyl}piperazin-1-yl) nicotinonitrile | White powder, 37 mg, 21% |
| NMR | 8.39(m, 1H); 8.09(m, 1H); 7.98(m, 1H); 7.61(m, 1H); 7.48(m, 2H); 6.58(d, 1H); | | |
| | 4.25(q, 1H); 3.71(m, 4H); 2.70(m, 4H); 1.66(d, 3H) | | |
| 15.15 | 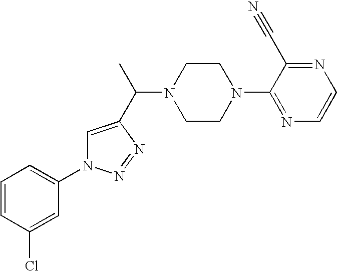 | 3-(4-{1-[1-(3-chlorophenyl)-1H-1,2,3- triazol-4-yl]ethyl}piperazin-1-yl) pyrazine-2-carbonitrile | Yellow solid, 17.2 mg, 12% |
| NMR | 8.24(s, 1H); 8.00(t, 1H); 7.88(m, 1H); 7.81(m, 1H); 7.67(m, 1H); 7.49(m, 1H); | | |
| | 4.11(q, 1H); 3.87(m, 4H); 2.72(m, 4H); 1.58(d, 3H) | | |
| 15.16 | 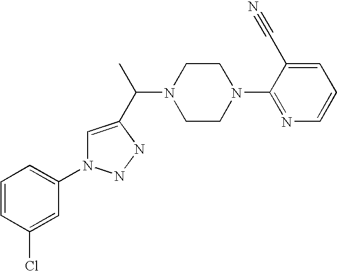 | 2-(4-{1-[1-(3-chlorophenyl)-1H-1,2,3- triazol-4-yl]ethyl}piperazin-1-yl) nicotinonitrile | Yellow oil, 28.5 mg, 36% |
| NMR | 8.24(m, 1H); 7.99(m, 1H); 7.81(s, 1H); 7.68(m, 1H); 7.67(m, 1H); 7.45(m, 2H); | | |
| | 4.13(q, 1H); 3.86(m, 4H); 2.72(m, 4H); 1.57(d, 3h) | | |
| 15.17 | 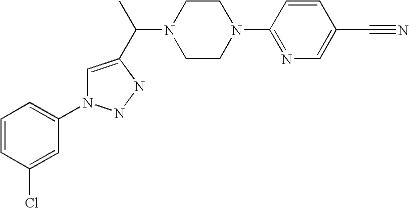 | 6-(4-{1-[1-(3-chlorophenyl)-1H-1,2,3- triazol-4-yl]ethyl}piperazin-1-yl) nicotinonitrile | Yellow oil, 28.8 mg, 36% |
| NMR | 8.39(m, 1H); 7.88(s, 1H); 7.79(s, 1H); 7.60(m, 1H); 7.57(m, 1H); 7.44(m, 2H); | | |
| | 6.58(d, 1H); 4.11(m, 1H); 3.71(m, 4H); 2.64(m, 4H); 1.58(d, 3H) | | |
| 15.18 | 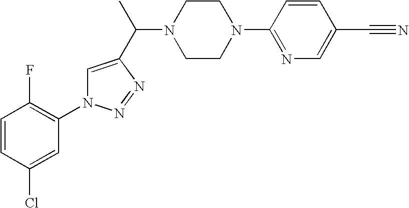 | 6-(4-{1-[1-(5-chloro-2-fluoro phenyl)- 1H-1,2,3-triazol-4-yl]ethyl}piperazine-1-yl) nicotinonitrile | Off-white solid, 12.1 mg, 15.7% |
| NMR | 8.39(s, 1H); 8.06(m, 1H); 8.00(s, 1H); 7.60(m, 1H); 7.28(m, 1H); 7.24(3, 1H); | | |
| | 6.59(d, 1H); 4.12(m, 1H); 3.69(m, 4H); 2.60(m, 4H); 1.57(m, 3H) | | |
| 15.19 | 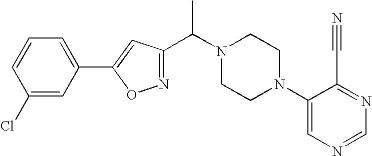 | 5-(4-{1-[5-(3-chlorophenyl)isoxazol- 3-yl]ethyl}piperazin-1-yl)pyrimidine- 4-carbonitrile | Yellow oil, 15.4 mg, 5.3% |
| NMR | 8.81(s, 1H); 8.57(s, 1H); 7.78(m, 1H); 7.69(m, 1H); 7.42(m, 2H); 6.55(s, 1H); | | |
| | 3.95(q, 1H); 3.44(m, 4H); 2.78(m, 4H); 1.51(d, 3H) | | |
| 15.20 | 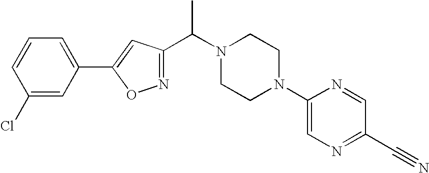 | 5-(4-{1-[5-(3-chlorophenyl)isoxazol- 3-yl]ethyl}piperazin-1-yl)pyrazine-2- carbonitrile | Yellow oil, 39.8 mg, 14% |

| | |
|---|---|
| NMR | 8.31(s, 1H); 8.10(s, 1H); 7.76(m, 1H); 7.67(m, 1H); 7.42(m, 2H); 6.56(s, 1H); |
| | 3.93(q, 1H); 3.76(m, 4H); 2.66(m, 4H); 1.50(d, 3H) |

The following compounds were made in the same manner from the corresponding chloride instead of the mesylate.

| Example | Structure | Name | Yield |
|---|---|---|---|
| 15.21 | 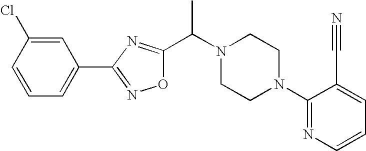 | 2-(4-{1-[3-(3-chlorophenyl)-1,2,4- oxadiazol-5-yl]ethyl}piperazin-1-yl) nicotinonitrile | Yellow oil, 3.3 mg |
| NMR | 8.34(m, 1H); 8.12(m, 1H); 8.00(m, 1H); 7.77(m, 1H); 7.48(m, 2H); 6.75(m, 1H); | | |
| | 4.25(q, 1H); 3.77(m, 4H); 2.70(m, 4H); 1.66(d, 3H) | | |
| 15.22 | 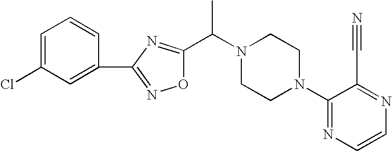 | 3-(4-{1-[3-(3-chlorophenyl)-1,2,4- oxadiazol-5-yl]ethyl}piperazin-1-yl) pyrazine-2-carbonitrile | Yellow oil, 4.8 mg |

| | |
|---|---|
| NMR | 8.25(m, 1H); 8.12(m, 1H); 8.01(m, 1H); 7.50(m, 1H); 4.26(q, 1H); 3.88(m, 4H); |
| | 2.85(m, 2H); 2.75(m, 2H); 1.66(d, 3H) |

### EXAMPLE 16

### GENERAL PROCEDURE: Reductive Amination with Aldehyde

Sodium cyanoborohydride (1M THF) (1 mmol) was added to a solution of arylpiperazine (1 mmol), acetic acid (0.09 ml) and heterocyclic aldehyde (1 mmol) in MeOH (4.5 ml). The reaction mixture was left to stir at RT overnight. Saturated sodium bicarbonate was added and the product was extracted with DCM. The organic phase was isolated, washed with water and brine, dried over anhydrous sodium sulfate, and concentrated in vacuo. SPE column chromatography (silica gel, 0-50% ethyl acetate in hexanes) yielded the desired compound.

The following compounds were synthesized using the above procedure.

| Example | Structure | Name | Yield |
|---|---|---|---|
| 16.1 | 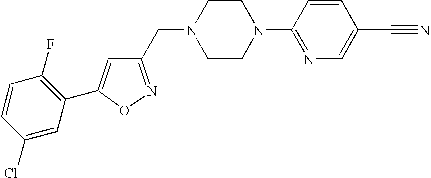 | 6-(4-{[5-(5-chloro-2-fluorophenyl) isoxazol-3-yl]ethyl}piperazin-1-yl) nicotinonitrile | yellow oil, 45 mg, 51% |
| NMR | 8.41(m, 1H); 7.95(m, 1H); 7.62(m, 1H); 7.28(m, 1H); 7.17(m, 1H); 6.81(d, 1H); | | |
| | 6.61(m, 1H); 3.74(m, 6H); 2.64(m, 4H) | | |
| 16.2 | 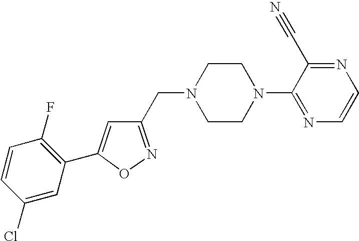 | 3-(4-{[5-(5-chloro-2-fluorophenyl) isoxazol-3-yl]ethyl}piperazin-1-yl) pyrazine-2-carbonitrile | yellow oil, 40 mg, 52.5% |

| | |
|---|---|
| NMR | 8.27(m, 1H); 8.03(m, 1H); 7.96(m, 1H); 7.40(m, 1H); 7.17(m, 1H); 6.81(d, 1H); |
| | 3.88(m, 4H); 3.76(s, 2H); 2.74(m, 4H) |

### EXAMPLE 17.1

### Amide via EDCI Coupling of Acid to Aryl Piperazine

### 1-{[-(3-chlorophenyl)isoxazol-3-yl]carbonyl}-4-(3-nitropyridin-2-yl)piperazine

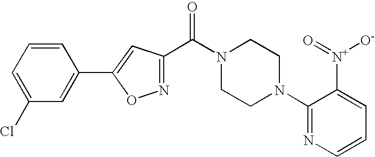

A mixture of 1-(3-nitropyridin-2-yl)piperazine (43.7 mg, 0.2 mmol), 5-(3-chlorophenyl)isoxazole-3-carboxylic acid (44.7 mg, 0.21 mmol), EDCI (38.3 mg, 0.2 mmol) and HOBt (27.0 mg, 0.2 mmol) were stirred in DMF (1 mL) at RT overnight. The mixture was diluted with water and extracted into DCM. The organic extract was dried, filtered and concentrated in vacuo. The resulting solid was triturated with ether to give the title compound (75.7 mg, 91.4%, yellow solid). ¹H NMR (CDCl₃) δ (ppm): 8.41 (dd, 1H), 8.22 (dd, 1H), 7.82 (t, 1H), 7.70 (dd, 1H), 7.47 (m, 2H), 6.92 (s, 1H), 6.88 (dd, 1H), 4.15 (m, 2H), 3.99 (m, 2H), 3.60 (m, 4H).

The following compound was prepared in this manner

| Example | Structure | Name | Yield |
|---|---|---|---|
| 17.2 | 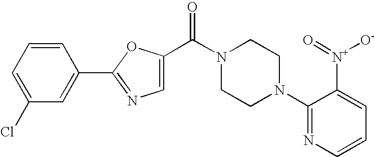 | 1-{[2-(3-chlorophenyl)-1,3- oxazol-5-yl]carbonyl}-4-(3- nitropyridin-2-yl)piperazine | yellow solid, 73.1 mg, 88.7% |

| | |
|---|---|
| NMR | 8.4(dd, 1H), 8.3(s, 1H), 8.21(dd, 1H), 8.05(m, 1H), 7.95(d, 1H), 7.47(m, 2H), |
| | 6.86(dd, 1H), 4.4(m, 2H), 3.95(m, 2H), 3.64(m, 4H) |

### EXAMPLE 18

### Pharmaceutical Examples

Functional Assessment of mGluR5 Antagonism in Cell Lines Expressing mGluR5D

The properties of the compounds of the invention can be analyzed using standard assays for pharmacological activity. Examples of glutamate receptor assays are well known in the art as described in for example Aramori et al., Neuron 8:757 (1992), Tanabe et al., Neuron 8:169 (1992), Miller et al., J. Neuroscience 15: 6103 (1995), Balazs, et al., J. Neurochemistry 69:151 (1997). The methodology described in these publications is incorporated herein by reference. Conveniently, the compounds of the invention can be studied by means of an assay (FLIPR) that measures the mobilization of intracellular calcium, [Ca²⁺]ᵢ in cells expressing mGluR5 or another assay (IP3) that measures inositol phosphate turnover.

FLIPR Assay

Cells expressing human mGluR5d as described in WO97/05252 are seeded at a density of 100,000 cells per well on collagen coated clear bottom 96-well plates with black sides and experiments are done 24 h following seeding. All assays are done in a buffer containing 127 mM NaCl, 5 mM KCl, 2 mM MgCl₂, 0.7 mM NaH₂PO₄, 2 mM CaCl₂, 0.422 mg/ml NaHCO₃, 2.4 mg/ml HEPES, 1.8 mg/ml glucose and 1 mg/ml BSA Fraction IV (pH 7.4). Cell cultures in the 96-well plates are loaded for 60 min. in the above mentioned buffer containing 4 μM of the acetoxymethyl ester form of the fluorescent calcium indicator fluo-3 (Molecular Probes, Eugene, Oreg.) in 0.01% pluronic acid (a proprietary, non-ionic surfactant polyol—CAS Number 9003-11-6). Following the loading period the fluo-3 buffer is removed and replaced with fresh assay buffer. FLIPR experiments are done using a laser setting of 0.800 W and a 0.4 second CCD camera shutter speed with excitation and emission wavelengths of 488 nm and 562 nm, respectively. Each experiment is initiated with 160 μl of buffer present in each well of the cell plate. A 40 μl addition from the antagonist plate was followed by a 50 μL addition from the agonist plate. A 90 second interval separates the antagonist and agonist additions. The fluorescence signal is sampled 50 times at 1 second intervals followed by 3 samples at 5 second intervals immediately after each of the two additions. Responses are measured as the difference between the peak height of the response to agonist, less the background fluorescence within the sample period. IC₅₀ determinations are made using a linear least squares fitting program.

IP3 Assay

An additional functional assay for mGluR5d is described in WO97/05252 and is based on phosphatidylinositol turnover. Receptor activation stimulates phospholipase C activity and leads to increased formation of inositol 1,4,5,triphosphate (IP₃).

GHEK stably expressing the human mGluR5d are seeded onto 24 well poly-L-lysine coated plates at 40×10⁴ cells/well in media containing 1 μCi/well [3H] myo-inositol. Cells were incubated overnight (16 h), then washed three times and incubated for 1 h at 37° C. in HEPES buffered saline (146 mM NaCl, 4.2 mM KCl, 0.5 mM MgCl₂, 0.1% glucose, 20 mM HEPES, pH 7.4) supplemented with 1 unit/ml glutamate pyruvate transaminase and 2 mM pyruvate. Cells are washed once in HEPES buffered saline and pre-incubated for 10 min in HEPES buffered saline containing 10 mM LiCl. Compounds are incubated in duplicate at 37° C. for 15 min, then either glutamate (80 μM) or DHPG (30 μM) is added and incubated for an additional 30 min. The reaction is terminated by the addition of 0.5 ml perchloric acid (5%) on ice, with incubation at 4° C. for at least 30 min. Samples are collected in 15 ml polyproplylene tubes and inositol phosphates are separated using ion-exchange resin (Dowex AG1-X8 formate form, 200-400 mesh, BIORAD) columns. Inositol phosphate separation was done by first eluting glycero phosphatidyl inositol with 8 ml 30 mM ammonium formate. Next, total inositol phosphates is eluted with 8 ml 700 mM ammonium formate/100 mM formic acid and collected in scintillation vials. This eluate is then mixed with 8 ml of scintillant and [3H] inositol incorporation is determined by scintillation counting. The dpm counts from the duplicate samples are plotted and IC₅₀ determinations are generated using a linear least squares fitting program.

Generally, the compounds of the present invention were active in the assays described herein at concentrations (or with EC₅₀ values) of less than 10 μM. Preferred compounds of the invention have EC₅₀ values of less than 1 μM; more preferred compounds of less than about 100 nM. For example, the compounds of Examples 16.1, 15.11, 15.16 and 15.17 have IC₅₀ values of 199, 101, 1082 and 159 nM, respectively.

What is claimed is:

1. A compound of formula I: 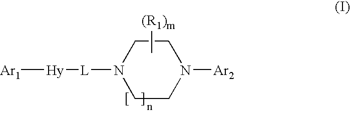
wherein:
Ar₁ and Ar₂ are independently selected, optionally substituted, aryl or heteroaryl groups, wherein the substituents are selected from the group consisting of F, Cl, Br, I, OH, nitro, C₁₋₆-alkyl, C₁₋₆-alkylhalo, OC₁₋₆-alkyl, OC₁₋₆-alkylhalo, C₂₋₆-alkenyl, C₂₋₆-alkynyl, CN, CO₂R², SR², S(O)R², SO₂R², aryl, heteroaryl, cycloalkyl and heterocycloalkyl, wherein any cyclic substituent may be further substituted with at least one substituent selected from the group consisting of F, Cl, Br, I, OH, nitro, C₁₋₆-alkyl, C₁₋₆-alkylhalo, OC₁₋₆-alkyl, OC₁₋₆-alkylhalo, C₂₋₆-alkenyl, C₂₋₆-alkynyl, CN, CO₂R², SR², S(O)R² and SO₂R²;
R₁, in each instance, is independently selected from the group consisting of F, Cl, Br, I, OH, CN, nitro, C₁₋₆-allyl, OC₁₋₆-alkyl, C₁₋₆-alkylhalo, OC₁₋₆-alkylhalo, (CO)R², O(CO)R², O(CO)OR², CO₂R, CONR²R³, C₁₋₆-alkyleneOR², OC₂₋₆-alkyleneOR² and C₁₋₆-alkylenecyano;
R² and R³ are independently selected from the group consisting of H, C₁₋₆-alkyl, C₁₋₆-alkylhalo, C₂₋₆-alkenyl, C₂₋₆-alkynyl and cycloalkyl;
Hy is a 5-membered heterocyclic ring containing two or three heteroatoms independently selected from the group consisting of N, O and S, wherein the ring is optionally substituted with one or more substituents selected from the group consisting of F, Cl, Br, I, OH, nitro, C₁₋₆-alkyl, C₁₋₆-alkylhalo, OC₁₋₆-alkyl, OC₁₋₆-alkylhalo, CN, CO₂R², CONR²R³, SR², S(O)R² and SO₂R²;
L is selected from the group consisting of —CR⁴R⁵—, —C(O)—, —C(NR⁴)— and —C(S)—;
R⁴ and R⁵ are independently selected from the group consisting of H, C₁₋₆-alkyl, C₁₋₆-alkylhalo, C₂₋₆-alkenyl and C₂₋₆-alkynyl;
m is an integer selected from the group consisting of 0, 1, 2, 3 and 4; and
n is an integer selected from the group consisting of 1 and 2;
or a pharmaceutically acceptable salt, hydrate, solvate, isoform, tautomer, optical isomer, or combination thereof

2. A compound according to claim 1, wherein Ar₁ is an optionally-substituted phenyl group.

3. A compound according to claim 2 wherein Ar₂ is selected from the group consisting of an optionally-substituted pyridyl group and an optionally-substituted pyrazine group.

4. A compound according to claim 3 wherein Ar₂ is an optionally-substituted 2-pyridyl group.

5. A compound according to claim 4 wherein L is selected from the group consisting of CH₂ and CH(Me).

6. A compound according to claim 5 wherein Hy is selected from the group consisting of isoxazole, 1,2,4-oxadiazole and 1,2,3-triazole.

7. A compound selected from the group consisting of:
3-(4-{1-[5-(3-chlorophenyl)isoxazol-3-yl]ethyl}piperazin-1-yl)pyrazine-2-carbonitrile,
2-(4-{1-[5-(3-chlorophenyl)isoxazol-3-yl]ethyl}piperazin-1-yl)nicotinonitrile,
6-(4-{1-[5-(3-chlorophenyl)isoxazol-3-yl]ethyl}piperazin-1-yl)nicotinonitrile,
1-{1-[5-(3-chlorophenyl)isoxazol-3-yl]ethyl}-4-pyridin-2-ylpiperazine,
2-(4-{1-[5-(3-chlorophenyl)isoxazol-3-yl]ethyl}piperazin-1-yl)pyrazine,
3-(4-{1-[5-(3-cyanophenyl)isoxazol-3-yl]ethyl}piperazin-1-yl)pyrazine-2-carbonitrile,
3-(4-{1-[5-(5-chloro-2-fluorophenyl)isoxazol-3-yl]ethyl}piperazin-1-yl)pyrazine-2-carbonitrile,
6-(4-{1-[5-(5-chloro-2-fluorophenyl)isoxazol-3-yl]ethyl}piperazin-1-yl)nicotinonitrile,
3-(3-{1-[4-(3-nitropyridin-2-yl)piperazin-1-yl]ethyl}isoxazol-5-yl)benzonitrile,
1-{1-[5-3-chlorophenyl)isoxazol-3-yl]ethyl}-4-(3-nitropyridin-2-yl)piperazine,
3-(4-{1-[5-3-chlorophenyl)-1,2,4-oxadiazol-3-yl]ethyl}piperazin-1-yl)pyrazine-2-carbonitrile,
6-(4-{1-[5-(3-chlorophenyl)-1,2,4-oxadiazol-3-yl]ethyl}piperazin-1-yl)nicotinonitrile
2-(4-{1-[5-(3-chlorophenyl)-1,2,4-oxadiazol-3-yl]ethyl}piperazin-1-yl)nicotinonitrile
6-(4-{1-[3-(3-chlorophenyl)-1,2,4-oxadiazol-5-yl]ethyl}piperazin-1-yl)nicotinonitrile
3-(4-{1-[1-(3-chlorophenyl)-1H-1,2,3-triazol-4-yl]ethyl}piperazin-1-yl)pyrazine-2-carbonitrile,
2-(4-{1-[1-(3-chlorophenyl)-1H-1,2,3-triazol-4-yl]ethyl}piperazin-1-yl)nicotinonitrile,
6-(4-{1-[1-(3-chlorophenyl)-1H-1,2,3-triazol-4-yl]ethyl}piperazin-1-yl)nicotinonitrile,
6-(4-{1-[1-(5-chloro-2-fluoro phenyl)-1H-1,2,3-triazol-4-yl]ethyl}piperazine-1-yl)nicotinonitrile,
5-(4-{1-[5-(3-chlorophenyl)isoxazol-3-yl]ethyl}piperazin-1-yl)pyrimidine-4-carbonitrile,
5-(4-{1-[5-3-chlorophenyl)isoxazol-3-yl]ethyl}piperazin-1-yl)pyrazine-2-carbonitrile,
2-(4-{1-[3-(3-chlorophenyl)-1,2,4-oxadiazol-5-yl]ethyl}piperazin-1-yl)nicotinonitrile,
3-(4-{1-[3-(3-chlorophenyl)-1,2,4-oxadiazol-5-yl]ethyl}piperazin-1-yl)pyrazine-2-carbonitrile,
6-(4-{[5-(5-chloro-2-fluorophenyl)isoxazol-3-yl]methyl}piperazin-1-yl)nicotinonitrile,
3-(4-{[5-(5-chloro-2-fluorophenyl)isoxazol-3-yl]methyl}piperazin-1-yl)pyrazine-2-carbonitrile,
1-{[5-(3-chlorophenyl)isoxazol-3-yl]carbonyl}-4-nitropyridin-2-yl)piperazine, and
1-{[2-(3-chlorophenyl)-1,3-oxazol-5-yl]carbonyl}-4-(3-nitropyridin-2-yl)piperazine.

8. A pharmaceutical composition comprising as active ingredient a therapeutically effective amount of the compound according to claim 1, in association with one or more pharmaceutically acceptable diluents, excipients and/or inert carriers.

9. The pharmaceutical composition according to claim 8 for use in the treatment of mGluR 5 mediated disorders.

10. The compound according to claim 1 for use in therapy.

11. The compound according to claim 1 for use in treatment of mGluR5 mediated disorders.

12. Use of the compound according to claim 1 in the manufacture of a medicament for the treatment of mGluR5-mediated disorders.

13. A method of treatment of mGluR5-mediated disorders, comprising administering to a mammal a therapeutically effective amount of the compound according to claim 1.

14. The method according to claim 13, wherein the mammal is a human.

15. The method according to claim 14, wherein the disorders are neurological disorders.

16. The method according to claim 14, wherein the disorders are psychiatric disorders.

17. The method according to claim 14, wherein the disorders are chronic and acute pain disorders.

18. The method according to claim 14, wherein the disorders are gastrointestinal disorders.

19. A method for inhibiting activation of mGlur5 receptors, comprising treating a cell containing said receptor with an effective amount of a compound according to claim 1.
